(19) United States
(12) Patent Application Publication
(10) Pub. No.: US 2018/0125992 A1
(43) Pub. Date: May 10, 2018

(54) DRUG DELIVERY CONJUGATES OF TERTIARY AMINE CONTAINING DRUGS

(71) Applicant: ENDOCYTE, INC., West Lafayette, IN (US)

(72) Inventors: Iontcho Radoslavov VLAHOV, West Lafayette, IN (US); Fei YOU, West Lafayette, IN (US); Christopher Paul LEAMON, West Lafayette, IN (US)

(21) Appl. No.: 15/798,760

(22) Filed: Oct. 31, 2017

Related U.S. Application Data
(60) Provisional application No. 62/417,798, filed on Nov. 4, 2016.

(51) Int. Cl. A61K 47/65; A61K 47/54; A61K 38/05

(52) U.S. Cl. CPC A61K 47/65; A61K 38/05; A61K 47/545

(57) ABSTRACT

The present disclosure relates to conjugates of tertiary amine containing drugs. The present disclosure also relates to pharmaceutical compositions of the conjugates described herein, methods of making, and methods of using the same.

### CROSS REFERENCE TO RELATED APPLICATIONS

This application claims priority under 35 U.S.C. § 119 to U.S. Provisional Application Ser. No. 62/417,798 entitled “DRUG DELIVERY CONJUGATES OF TERTIARY AMINE CONTAINING DRUGS,” which was filed on Nov. 4, 2016, and is expressly incorporated herein by reference.

### TECHNICAL FIELD

The present disclosure relates to conjugates of tertiary amine containing drugs. The present disclosure also relates to pharmaceutical compositions of the conjugates described herein, methods of making, and methods of using the same.

### BACKGROUND

The mammalian immune system provides a means for the recognition and elimination of pathogenic cells, such as tumor cells, and other invading foreign pathogens. While the immune system normally provides a strong line of defense, there are many instances where pathogenic cells, such as cancer cells, and other infectious agents evade a host immune response and proliferate or persist with concomitant host pathogenicity. Chemotherapeutic agents and radiation therapies have been developed to eliminate, for example, replicating neoplasms. However, many of the currently available chemotherapeutic agents and radiation therapy regimens have adverse side effects because they lack sufficient selectivity to preferentially destroy pathogenic cells, and therefore, may also harm normal host cells, such as cells of the hematopoietic system, and other non-pathogenic cells. The adverse side effects of these anticancer drugs highlight the need for the development of new therapies selective for pathogenic cell populations and with reduced host toxicity.

Researchers have developed therapeutic protocols for destroying pathogenic cells by targeting cytotoxic compounds to such cells. Many of these protocols utilize toxins conjugated to antibodies that bind to antigens unique to or overexpressed by the pathogenic cells in an attempt to minimize delivery of the toxin to normal cells. Using this approach, certain immunotoxins have been developed consisting of antibodies directed to specific antigens on pathogenic cells, the antibodies being linked to toxins such as ricin, Pseudomonas exotoxin, Diptheria toxin, and tumor necrosis factor. These immunotoxins target pathogenic cells, such as tumor cells, bearing the specific antigens recognized by the antibody (Olsnes, S., Immunol. Today, 10, pp. 291-295, 1989; Melby, E. L., Cancer Res., 53(8), pp. 1755-1760, 1993; Better, M.D., PCT Publication Number WO 91/07418, published May 30, 1991).

Another approach for targeting populations of pathogenic cells, such as cancer cells or foreign pathogens, in a host is to enhance the host immune response against the pathogenic cells to avoid the need for administration of compounds that may also exhibit independent host toxicity. One reported strategy for immunotherapy is to bind antibodies, for example, genetically engineered multimeric antibodies, to the surface of tumor cells to display the constant region of the antibodies on the cell surface and thereby induce tumor cell killing by various immune-system mediated processes (De Vita, V. T., Biologic Therapy of Cancer, 2d ed. Philadelphia, Lippincott, 1995; Soulillou, J. P., U.S. Pat. No. 5,672,486). However, these approaches have been complicated by the difficulties in defining tumor-specific antigens.

The delivery of therapeutic agents through ligand-drug conjugates has become an area of great interest in recent years with numerous approaches being pursued. One such approach is based in the fact that folate plays important roles in nucleotide biosynthesis and cell division, intracellular activities which occur in both malignant and certain normal cells. The folate receptor has a high affinity for folate, which, upon binding the folate receptor, impacts the cell cycle in dividing cells. As a result, folate receptors have been implicated in a variety of cancers (e.g., ovarian, endometrial, lung and breast) which have been shown to demonstrate high folate receptor expression. In contrast, folate receptor expression in normal tissues is limited (e.g., kidney, liver, intestines and placenta). This differential expression of the folate receptor in neoplastic and normal tissues makes the folate receptor an ideal target for small molecule drug development. The development of folate conjugates has recently been one avenue for the discovery of new treatments that take advantage of differential expression of the folate receptor (U.S. Pat. No. 7,601,332). Due to the structural complexity of the ligand-drug conjugates developed to date, there exists a great need to develop ligand-drug conjugates that possess superior properties based on the structure of the ligand, linker, and/or drug, and develop novel approaches to delivery of an ever increasing variety of drugs to a target cell.

### SUMMARY

It has been discovered that certain tertiary amine containing drugs are amenable to conjugation to a targeting ligand through a linkage that occurs at the nitrogen atom of a tertiary amine in tertiary amine drugs to provide a conjugate comprising a quaternary amine group.

In one illustrative embodiment, the disclosure provides a drug conjugate comprising a binding ligand, a linker, and a tertiary amine containing drug, wherein the linker comprises a disulfide bond, the binding ligand is covalently attached to the linker, and the tertiary amine containing drug is covalently attached to the linker through a nitrogen atom of a tertiary amine group on the tertiary amine containing drug, such that the drug conjugate contains a quaternary amine.

In another illustrative embodiment, the disclosure provides a pharmaceutical composition comprising a drug conjugate as described herein, and optionally at least one excipient.

In another illustrative embodiment, the disclosure provides a method of treating disease comprising administering a therapeutically effective amount of a drug conjugate as described herein. In some aspects of these embodiments, the disease is a cancer described herein.

In another illustrative embodiment, the disclosure provides a drug conjugate comprising a binding ligand, a linker, and a tertiary amine containing drug, wherein the linker comprises a disulfide bond, the binding ligand is covalently attached to the linker, and the tertiary amine containing drug is covalently attached to the linker through a nitrogen atom of a tertiary amine group on the tertiary amine containing drug, such that the drug conjugate contains a quaternary amine, for treating a disease in a patient. In some aspects of these embodiments, the disease is a cancer described herein.

In another illustrative embodiment, the disclosure provides for the use of drug conjugate comprising a binding ligand, a linker, and a tertiary amine containing drug, wherein the linker comprises a disulfide bond, the binding ligand is covalently attached to the linker, and the tertiary amine containing drug is covalently attached to the linker through a nitrogen atom of a tertiary amine group on the tertiary amine containing drug, such that the drug conjugate contains a quaternary amine, in the preparation of a medicament for treating a disease in a patient. In some aspects of these embodiments, the disease is a cancer described herein.

Additional illustrative and non-limiting embodiments of the invention are described in the following enumerated clauses. All combinations of the following clauses are understood to be additional embodiments of the invention described herein. All applicable combinations of these embodiments with the embodiments described in the DETAILED DESCRIPTION section of the application are also embodiments of the invention.

1. A drug conjugate comprising a binding ligand, a linker, and a tertiary amine containing drug, wherein the linker comprises a disulfide bond, the binding ligand is covalently attached to the linker, and the tertiary amine containing drug is covalently attached to the linker through a nitrogen atom of a tertiary amine group on the tertiary amine containing drug, such that the drug conjugate contains a quaternary amine.

2. The drug conjugate of clause 1, or a pharmaceutically acceptable salt thereof, wherein linker comprises a moiety L¹ of the formula selected from the group consisting of

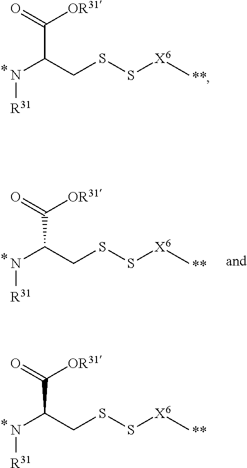

wherein

each of R³¹ and R^{31′} is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)NR³³R^{33′}, —NR³²S(O)₂NR³³R^{33′}, —C(O)R³², —C(O)OR³² or —C(O)NR³²R^{32′};

X⁶ is independently a C₁-C₆ alkyl, C₂-C₆ heteroalkyl or C₆-C₁₀ aryl, wherein each hydrogen atom in C₁-C₆ alkyl, C₁-C₆ heteroalkyl and C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};

each R³², R^{32′}, R³³, R^{33′}, R³⁴, R^{34′}, R³⁵ and R^{35′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

wherein ** is a covalent bond to a nitrogen atom of a tertiary amine on the tertiary amine containing drug; and * is a covalent bond to the rest of the drug conjugate.

3. The drug conjugate of clause 2, or a pharmaceutically acceptable salt thereof, wherein X⁶ is C₁-C₆ alkyl; wherein each hydrogen atom in C₁-C₆ alkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}.

4. The drug conjugate of clause 3, or a pharmaceutically acceptable salt thereof, wherein X⁶ is methyl, ethyl, n-propyl, iso-propyl, n-butyl, sec-butyl or n-pentyl.

5. The drug conjugate of clause 2, or a pharmaceutically acceptable salt thereof, wherein X⁶ is C₁-C₆ heteroalkyl; wherein each hydrogen atom in C₁-C₆ heteroalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}.

6. The drug conjugate of clause 5, or a pharmaceutically acceptable salt thereof, wherein C₁-C₆ heteroalkyl comprises one heteroatom selected from the group consisting of N, O and S.

7. The drug conjugate of clause 2, or a pharmaceutically acceptable salt thereof, wherein X⁶ is C₆-C₁₀ aryl, wherein each hydrogen atom in C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)NR³⁵R^{35′}, —C(O)R³⁴, —C(O)OR³⁴ or —C(O)NR³⁴R^{34′}.

8. The drug conjugate of clause 7, or a pharmaceutically acceptable salt thereof, wherein C₆-C₁₀ aryl is phenyl.

9. The drug conjugate of any one of the preceding clauses, or a pharmaceutically acceptable salt thereof, wherein the tertiary amine containing drug is selected from the group consisting of an opioid, an antibiotic, an antidepressant and a cancer therapeutic.

10. The drug conjugate of any one of the preceding clauses, or a pharmaceutically acceptable salt thereof, wherein the tertiary amine containing drug is selected from the group consisting of morphine, hydrocodone, oxycodone, codeine, mitragynol, vinblastine, vincristine, vindesine, vinorelbine, clindamycin, novobiocin, retapamulin, dimethylpipBOR, N,N-dimethylsitafloxacin, rifampin, azithromycin, venlafaxine, mirtazapine, escitalopram, porfiromycin, pamamycin 601, macromerine, tatreponerine 8, imatinib, aripiprazole, buprenorphine, sildenafil, quetiapine, methylphenidate, doxycycline, solifenacin, lidocaine, eszopiclone, and tubulysin.

11. The drug conjugate of any one of the preceding clauses, or a pharmaceutically acceptable salt thereof, wherein the linker comprises at least one AA selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

12. The drug conjugate of any one of the preceding clauses, or a pharmaceutically acceptable salt thereof, wherein the linkers comprises at least one AA selected from the group consisting of L-arginine, L-aspartic acid, L-cysteine, D-arginine, D-aspartic acid, and D-cysteine.

13. The drug conjugate of any one of the preceding clauses, or a pharmaceutically acceptable salt thereof, wherein the linker further comprises at least one spacer linker (L²) of the formula

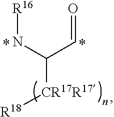

wherein

R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR², —S(O)R²², —S(O)₂R²², —S(O)NR²²R^{22′}, —S(O)₂NR²²R²², —OS(O)NR²²R^{22′}, —OS(O)₂NR²²R^{22′}, —NR²²R²², —NR²C(O)R²³, —NR²²C(O)OR²³, —NR²²C(O)NR²³R^{23′}, —NR²²S(O)R²³, —NR²²S(O)₂R²³, —NR²²S(O)NR²³R²³, —NR²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, (CH₂)ₚ(OCH₂CH₂)_{q}OR², —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

wherein each * is a covalent bond to the rest of the drug conjugate.

14. The drug conjugate of clause 13, or a pharmaceutically acceptable salt thereof, wherein L² is of the formula

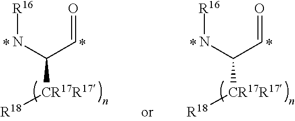

wherein each * is a covalent bond to the rest of the drug conjugate.

15. The drug conjugate of clause 13 or 14, or a pharmaceutically acceptable salt thereof, wherein R¹⁶ is H.

16. The drug conjugate of any one of clauses 13 to 15, or a pharmaceutically acceptable salt thereof, wherein R¹⁸ is selected from the group consisting of H, 5- to 7-membered heteroaryl, —OR²⁶, —NR²⁶C(O)R², —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR², —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

wherein each * is a covalent bond to the rest of the drug conjugate.

17. The drug conjugate of any one of clauses 13 to 15, or a pharmaceutically acceptable salt thereof, wherein R¹⁸ is selected from the group consisting of H, 5- to 7-membered heteroaryl, —OR²⁶, NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by —(CH₂)ₚOR², —OR²⁹, —(CH₂)ₚOS(O)₂OR²⁹ and —OS(O)₂OR²⁹,

each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;

R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);

R²⁸ is H or sugar;

n is 1, 2, 3, 4 or 5;

p is 1, 2, 3, 4 or 5; and

q is 1, 2, 3, 4 or 5;

wherein * is a covalent bond to the rest of the drug conjugate.

18. The drug conjugate of clause 13, or a pharmaceutically acceptable salt thereof, wherein each L² is independently selected from the group consisting of

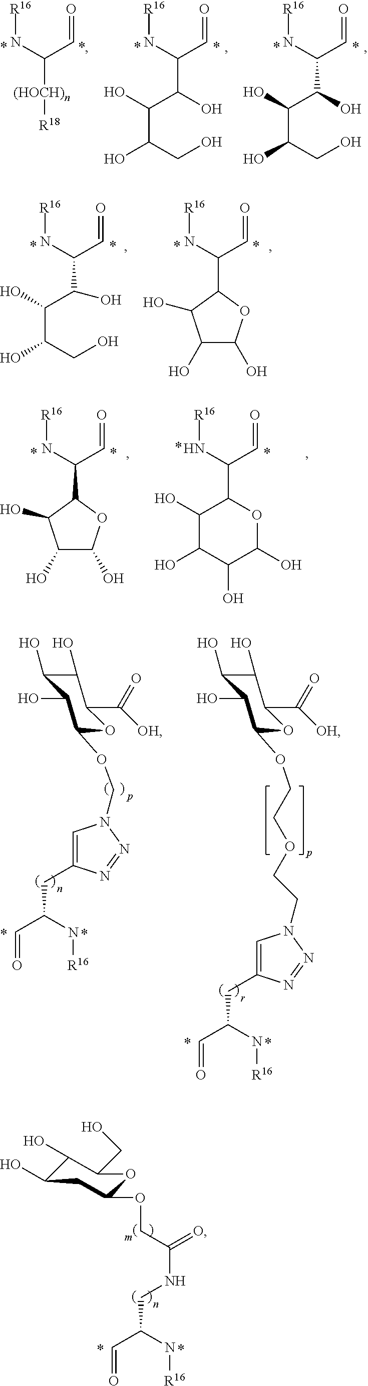

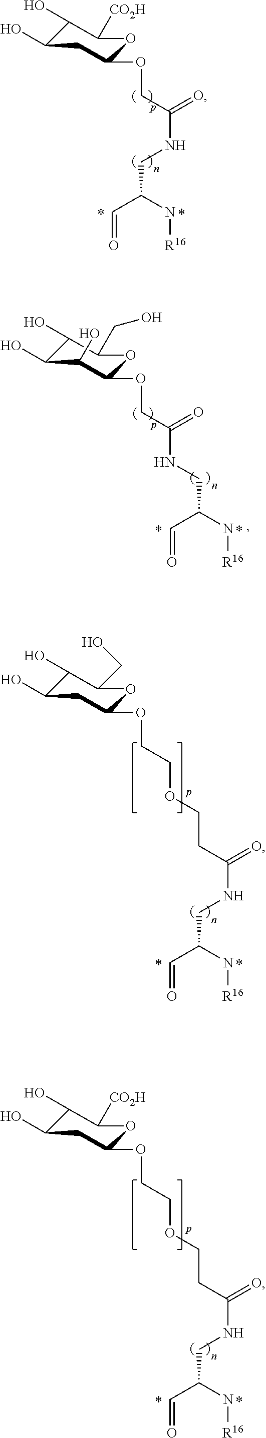

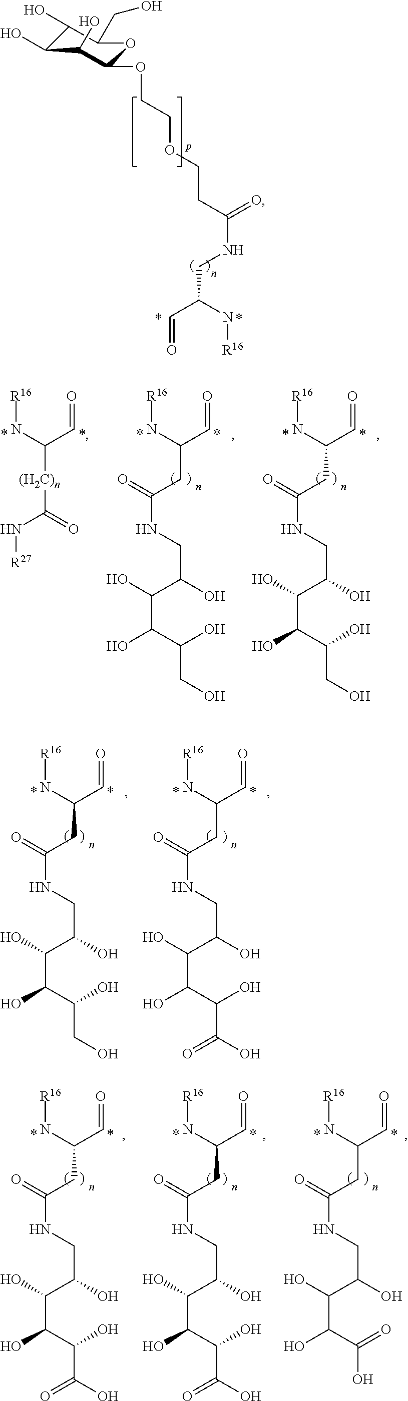

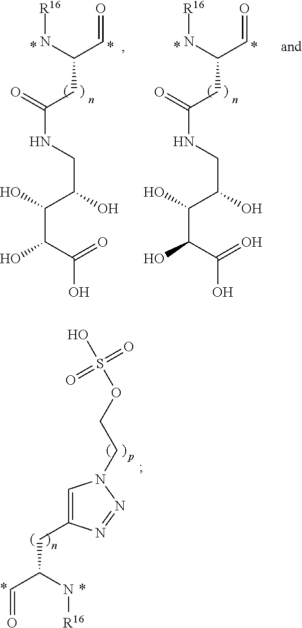

and combinations thereof,
wherein

R⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²S(O)R²¹, —NR²S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁷, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R², —NR²⁶C(O)OR²⁷, —NR²⁶C(O)NR²⁷R^{27′}, —NR²⁶C(═NR^{26″})NR²⁷R^{27′}, —NR²⁶S(O)R², —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26″}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR, —(CH₂)ₚ(OCH₂CH₂)_{q}OR⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each each R¹⁹, R^{19′}, R²⁰, R^{20′}, R²¹, R^{21′}, R²⁶, R^{26′}, R^{26″}, —R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

wherein each * is a covalent bond to the rest of the drug conjugate.

19. The drug conjugate of clause 13, or a pharmaceutically acceptable salt thereof, wherein each L² is selected from the group consisting of

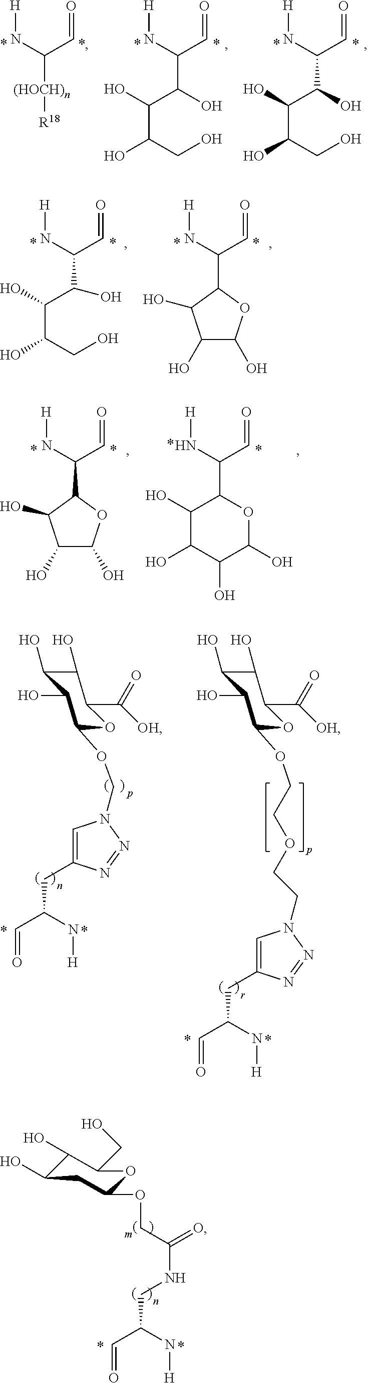

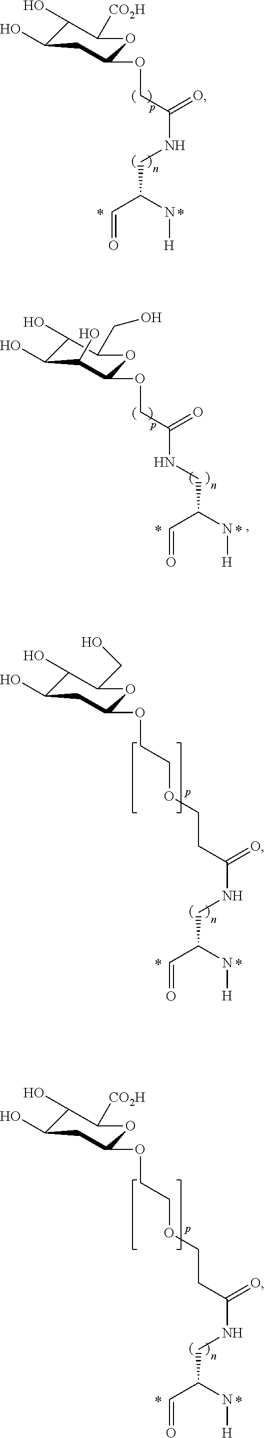

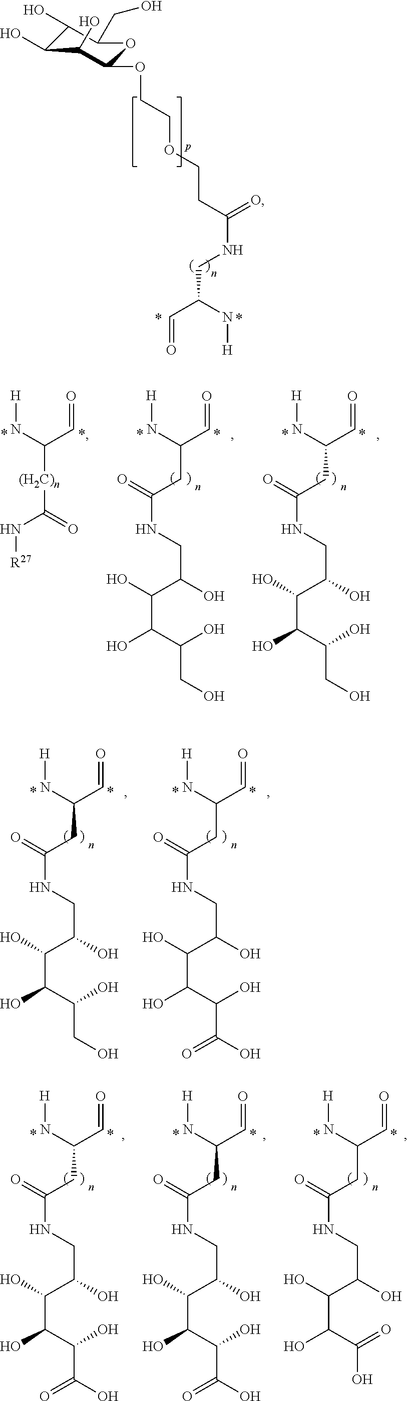

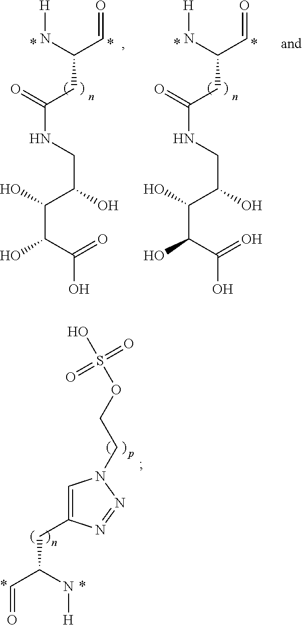

and combinations thereof,

wherein

R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R²⁹, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

wherein each * is a covalent bond to the rest of the drug conjugate.

20. The drug conjugate of any one of the preceding clauses, or a pharmaceutically acceptable salt thereof, wherein the binding ligand is of the formula

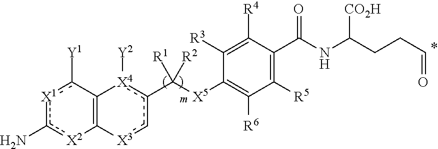

wherein

R¹ and R² in each instance are independently selected from the group consisting of H, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, C₁-C₆ alkyl, C₂-C₆ alkenyl or C₂-C₆ alkynyl;

X¹ is —NR¹¹—, ═N—, —N═, —C(R¹¹)═ or —C(R¹¹)—;

X² is —NR^{11′}— or ═N—;

X³ is —NR^{11″}—, —N═ or —C(R^{11′})═;

X⁴ is —N═ or —C═;

X⁵ is NR¹² or CR¹²R^{12′};

Y¹ is H, —OR¹³, —SR¹³ or —NR¹³R^{13′} when X¹ is —N═ or —C(R¹¹)═, or Y¹ is ═O when X¹ is —NR¹¹—, ═N— or —C(R¹¹)—;

Y² is H, C₁-C₆ alkyl, C₂-C₆ alkenyl, —C(O)R¹⁴, —C(O)OR¹⁴, —C(O)NR¹⁴R^{14′} when X⁴ is

—C═, or Y² is absent when X⁴ is —N═;

R¹¹, R^{11′}, R^{11″}, R¹², R^{12′}, R¹³, R^{13′}, R¹⁴ and R^{14′} are each independently selected from the group consisting of H, C₁-C₆ alkyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

wherein * is a covalent bond to the rest of the drug conjugate.

21. The drug conjugate of clause 20, wherein R¹ and R² are H.

22. The drug conjugate of clause 20 or 21, wherein m is 1.

23. The drug conjugate of any one of clauses 20 to 22, wherein R³ is H.

24. The drug conjugate of any one of clauses 20 to 23, wherein R⁴ is H.

25. The drug conjugate of any one of clauses 20 to 24, wherein R⁵ is H.

26. The drug conjugate of any one of clauses 20 to 25, wherein R⁶ is H.

27. The drug conjugate of any one of clauses 20 to 26, wherein X¹ is —NR¹¹, and R¹¹ is H.

28. The drug conjugate of any one of clauses 20 to 27, wherein X² is ═N—.

29. The drug conjugate of any one of clauses 20 to 28, wherein X³ is —N═.

30. The drug conjugate of any one of clauses 20 to 29, wherein X⁴ is —N═.

31. The drug conjugate of any one of clauses 20 to 30, wherein X⁵ is NR², and R¹² is H.

32. The drug conjugate of any one of clauses 20 to 31, wherein Y¹ is ═O.

33. The drug conjugate of any one of clauses 20 to 32, wherein Y² is absent.

34. The drug conjugate of clause 20, wherein B is of the formula

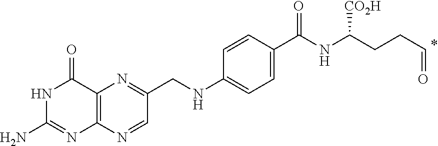 [SMILES: Nc1nc2ncc(CNc3ccc(C(=O)N[C@@H](CCC=O)C(=O)O)cc3)nc2c(=O)[nH]1]

wherein * is a covalent bond to the rest of the drug conjugate.

### BRIEF DESCRIPTION OF THE DRAWINGS

FIG. 1 shows UPLC/MS spectra for a release study of compound 1 with DTT or TCEP at room temperature: a. control; b. DTT release; c: TCEP release (neutral pH).

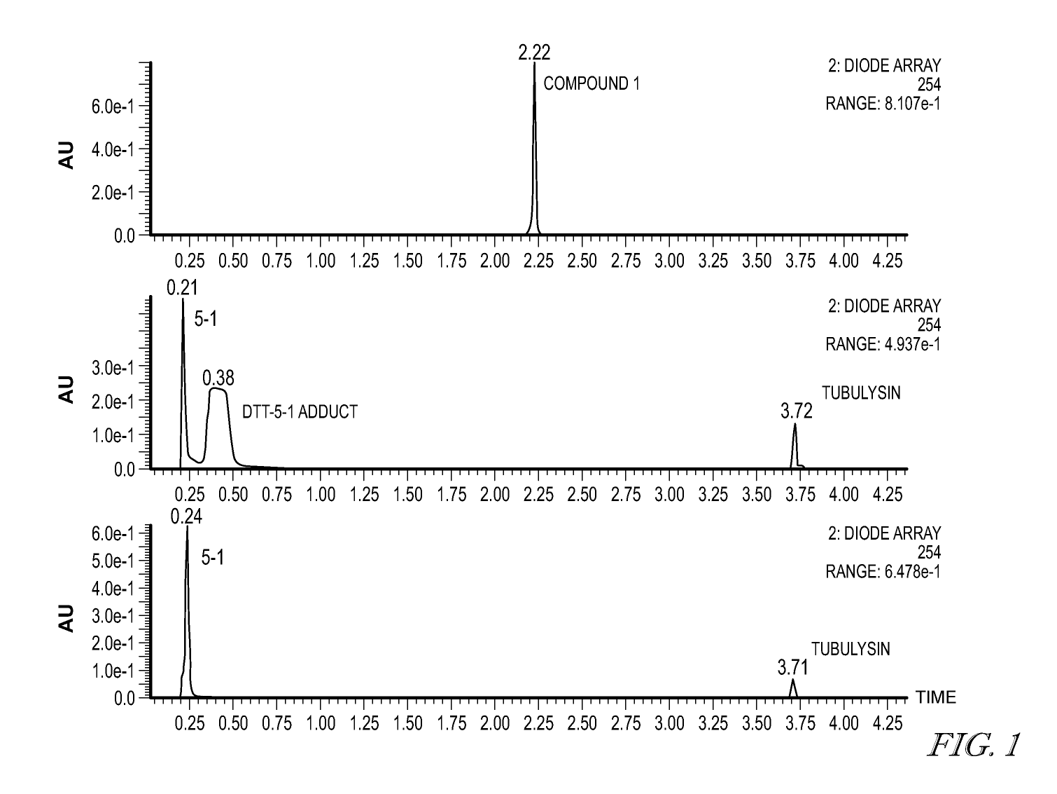

FIG. 2 shows plots of ³H-thymidine incorporation in KB cells of compound 1 (●) versus compound 1+excess folate (▪). Compound 1 showed an IC₅₀ of 5 nM.

FIG. 3 shows plots of ³H-thymidine incorporation in KB cells of compound 2 (●) versus compound 2+excess folate (▪). Compound 2 showed an IC₅₀ of 7 nM.

### DEFINITIONS

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e. C≡C). It will be understood that in certain embodiments alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkenyl may be unsubstituted, or substituted as described for alkyl or as described in the various embodiments provided herein. Illustrative alkenyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₆, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like.

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like.

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like.

As used herein, “hydroxy” or ““hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “aryloxy” refers to an —O-aryl or an —O-heteroaryl group. Representative examples include, but are not limited to, phenoxy, pyridinyloxy, furanyloxy, thienyloxy, pyrimidinyloxy, pyrazinyloxy, and the like, and the like.

As used herein, “mercapto” refers to an —SH group.

As used herein, “alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

As used herein, “arylthio” refers to an —S-aryl or an —S-heteroaryl group. Representative examples include, but are not limited to, phenylthio, pyridinylthio, furanylthio, thienylthio, pyrimidinylthio, and the like.

As used herein, “halo” or “halogen” refers to fluorine, chlorine, bromine or iodine.

As used herein, “trihalomethyl” refers to a methyl group having three halo substituents, such as a trifluoromethyl group.

As used herein, “cyano” refers to a —CN group.

As used herein, “sulfinyl” refers to a —S(O)R″ group, where R″ is any R group as described in the various embodiments provided herein, or R″ may be a hydroxyl group.

As used herein, “sulfonyl” refers to a —S(O)₂R″ group, where R″ is any R group as described in the various embodiments provided herein, or R″ may be a hydroxyl group.

As used herein, “S-sulfonamido” refers to a —S(O)₂NR″R″ group, where R″ is any R group as described in the various embodiments provided herein.

As used herein, “N-sulfonamido” refers to a —NR″S(O)₂R″ group, where R″ is any R group as described in the various embodiments provided herein.

As used herein, “O-carbamyl” refers to a —OC(O)NR″R″ group, where R″ is any R group as described in the various embodiments provided herein.

As used herein, “N-carbamyl” refers to an R″OC(O)NR″— group, where R″ is any R group as described in the various embodiments provided herein.

As used herein, “O-thiocarbamyl” refers to a —OC(S)NR″R″ group, where R″ is any R group as described in the various embodiments provided herein.

As used herein, “N-thiocarbamyl” refers to a R″OC(S)NR″— group, where R″ is any R group as described in the various embodiments provided herein.

As used herein, “amino” refers to an —NR″R″ group, where R″ is any R group as described in the various embodiments provided herein.

As used herein, “C-amido” refers to a —C(O)NR″R″ group, where R″ is any R group as described in the various embodiments provided herein.

As used herein, “N-amido” refers to a R″C(O)NR″— group, where R″ is any R group as described in the various embodiments provided herein.

As used herein, “nitro” refers to a —NO₂ group.

As used herein, “bond” refers to a covalent bond.

As used herein, “optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocycle group is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the term “pharmaceutically acceptable salt” refers to those salts which counter ions which may be used in pharmaceuticals. Such salts include:
(1) acid addition salts, which can be obtained by reaction of the free base of the parent conjugate with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent conjugate either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.
   Pharmaceutically acceptable salts are well known to those skilled in the art, and any such pharmaceutically acceptable salt may be contemplated in connection with the embodiments described herein

As used herein, “amino acid” (a.k.a. “AA”) means any molecule that includes an alpha-carbon atom covalently bonded to an amino group and an acid group. The acid group may include a carboxyl group. “Amino acid” may include molecules having one of the formulas:

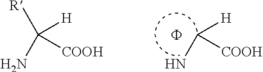

wherein R′ is a side group and Φ includes at least 3 carbon atoms. “Amino acid” includes stereoisomers such as the D-amino acid and L-amino acid forms. Illustrative amino acid groups include, but are not limited to, the twenty endogenous human amino acids and their derivatives, such as lysine (Lys), asparagine (Asn), threonine (Thr), serine (Ser), isoleucine (Ile), methionine (Met), proline (Pro), histidine (His), glutamine (Gln), arginine (Arg), glycine (Gly), aspartic acid (Asp), glutamic acid (Glu), alanine (Ala), valine (Val), phenylalanine (Phe), leucine (Leu), tyrosine (Tyr), cysteine (Cys), tryptophan (Trp), phosphoserine (PSER), sulfo-cysteine, arginosuccinic acid (ASA), hydroxyproline, phosphoethanolamine (PEA), sarcosine (SARC), taurine (TAU), carnosine (CARN), citrulline (CIT), anserine (ANS), 1,3-methyl-histidine (ME-HIS), alpha-amino-adipic acid (AAA), beta-alanine (BALA), ethanolamine (ETN), gamma-amino-butyric acid (GABA), beta-amino-isobutyric acid (BAIA), alpha-amino-butyric acid (BABA), L-allo-cystathionine (cystathionine-A; CYSTA-A), L-cystathionine (cystathionine-B; CYSTA-B), cystine, allo-isoleucine (ALLO-ILE), DL-hydroxylysine (hydroxylysine (I)), DL-allo-hydroxylysine (hydroxylysine (2)), ornithine (ORN), homocystine (HCY), and derivatives thereof. It will be appreciated that each of these examples are also contemplated in connection with the present disclosure in the D-configuration as noted above. Specifically, for example, D-lysine (D-Lys), D-asparagine (D-Asn), D-threonine (D-Thr), D-serine (D-Ser), D-isoleucine (D-Ile), D-methionine (D-Met), D-proline (D-Pro), D-histidine (D-His), D-glutamine (D-Gln), D-arginine (D-Arg), D-glycine (D-Gly), D-aspartic acid (D-Asp), D-glutamic acid (D-Glu), D-alanine (D-Ala), D-valine (D-Val), D-phenylalanine (D-Phe), D-leucine (D-Leu), D-tyrosine (D-Tyr), D-cysteine (D-Cys), D-tryptophan (D-Trp), D-citrulline (D-CIT), D-carnosine (D-CARN), and the like. In connection with the embodiments described herein, amino acids can be covalently attached to other portions of the conjugates described herein through their alpha-amino and carboxy functional groups (i.e. in a peptide bond configuration), or through their side chain functional groups (such as the side chain carboxy group in glutamic acid) and either their alpha-amino or carboxy functional groups. It will be understood that amino acids, when used in connection with the conjugates described herein, may exist as zwitterions in a conjugate in which they are incorporated.

As used herein, “sugar” refers to carbohydrates, such as monosaccharides, disaccharides, or oligosaccharides. In connection with the present disclosure, monosaccharides are preferred. Non-limiting examples of sugars include erythrose, threose, ribose, arabinose, xylose, lyxose, allose, altrose, glucose, mannose, galactose, ribulose, fructose, sorbose, tagatose, and the like. It will be understood that as used in connection with the present disclosure, sugar includes cyclic isomers of amino sugars, deoxy sugars, acidic sugars, and combinations thereof. Non-limiting examples of such sugars include, galactosamine, glucosamine, deoxyribose, fucose, rhamnose, glucuronic acid, ascorbic acid, and the like. In some embodiments, sugars for use in connection with the present disclosure include

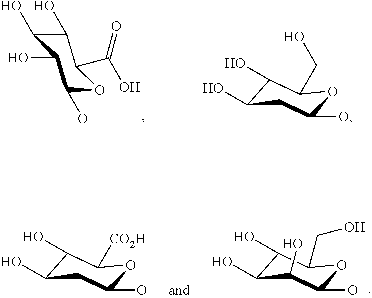

As used herein, “prodrug” refers to a compound that can be administered to a subject in a pharmacologically inactive form which then can be converted to a pharmacologically active form through a normal metabolic process, such as hydrolysis of an oxazolidine. It will be understood that the metabolic processes through which a prodrug can be converted to an active drug include, but are not limited to, one or more spontaneous chemical reaction(s), enzyme-catalyzed chemical reaction(s), and/or other metabolic chemical reaction(s), or a combination thereof. It will be appreciated that understood that a variety of metabolic processes are known in the art, and the metabolic processes through which the prodrugs described herein are converted to active drugs are non-limiting. A prodrug can be a precursor chemical compound of a drug that has a therapeutic effect on a subject.

As used herein, the term “releasable group” refers to a bond or bonds that can be broken (“a cleavable bond” or “cleavable bonds”) under physiological conditions, such as a pH-labile, acid-labile, base-labile, oxidatively labile, metabolically labile, biochemically labile, or enzyme-labile bond. It will be appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It will be appreciated that a releasable group can connect two adjacent atoms within a releasable linker and/or connect other linkers (e.g. AA, L¹, L², etc), B and/or D, as described herein. Alternatively, a releasable group can form part of a drug, D, and/or connect a drug, D, to other linkers (e.g. AA, L¹, L², etc) and/or B, as described herein. In the case where a releasable group connects two adjacent atoms within a releasable linker, following breakage of the cleavable bond, such releasable linker is broken into two or more fragments. Alternatively, in the case where a releaseable group connects a linker (e.g. AA, L¹, L², etc) to another moiety, such as another linker, a drug or binding ligand, then such releasable linker becomes separated from such other moiety following breaking of the cleavable bond or cleavable bonds. Alternatively, in the case where a releaseable group is within a drug, D, that is connected to a linker, another drug or a binding ligand, then following breaking of the cleavable bond or cleavable bonds, such linker, drug or binding ligand becomes separated from such drug or prodrug having the releaseable group within.

The lability of the releasable group can be adjusted by, for example, substituents at or near the cleavable bond, such as including alpha-branching adjacent to a cleavable disulfide bond, increasing the hydrophobicity of substituents on silicon in a moiety having silicon-oxygen bond that may be hydrolyzed, homologating alkoxy groups that form part of a ketal or acetal that may be hydrolyzed, and the like.

As used herein, the term “therapeutically effective amount” refers to an amount of a drug or pharmaceutical agent that elicits the biological or medicinal response in a subject (i.e. a tissue system, animal or human) that is being sought by a researcher, veterinarian, medical doctor or other clinician, which includes, but is not limited to, alleviation of the symptoms of the disease or disorder being treated. In one aspect, the therapeutically effective amount is that amount of an active which may treat or alleviate the disease or symptoms of the disease at a reasonable benefit/risk ratio applicable to any medical treatment. In another aspect, the therapeutically effective amount is that amount of an inactive prodrug which when converted through normal metabolic processes to produce an amount of active drug capable of eliciting the biological or medicinal response in a subject that is being sought.

It is also appreciated that the dose, whether referring to monotherapy or combination therapy, is advantageously selected with reference to any toxicity, or other undesirable side effect, that might occur during administration of one or more of the conjugates described herein. Further, it is appreciated that the co-therapies described herein may allow for the administration of lower doses of conjugates that show such toxicity, or other undesirable side effect, where those lower doses are below thresholds of toxicity or lower in the therapeutic window than would otherwise be administered in the absence of a cotherapy.

As used herein, “administering” includes all means of introducing the conjugates and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The conjugates and compositions described herein may be administered in unit dosage forms and/or formulations containing conventional nontoxic pharmaceutically-acceptable carriers, adjuvants, and/or vehicles.

As used herein “pharmaceutical composition” or “composition” refers to a mixture of one or more of the conjugates described herein, or pharmaceutically acceptable salts, solvates, hydrates thereof, with other chemical components, such as pharmaceutically acceptable excipients. The purpose of a pharmaceutical composition is to facilitate administration of a conjugate to a subject. Pharmaceutical compositions suitable for the delivery of conjugates described and methods for their preparation will be readily apparent to those skilled in the art. Such compositions and methods for their preparation may be found, for example, in ‘Remington's Pharmaceutical Sciences’, 19th Edition (Mack Publishing Company, 1995).

A “pharmaceutically acceptable excipient” refers to an inert substance added to a pharmaceutical composition to further facilitate administration of a conjugate such as a diluent or a carrier.

### DETAILED DESCRIPTION

In each of the foregoing and each of the following embodiments, it is to be understood that the formulae include and represent not only all pharmaceutically acceptable salts of the conjugates, but also include any and all hydrates and/or solvates of the conjugate formulae. It is appreciated that certain functional groups, such as the hydroxy, amino, and like groups form complexes and/or coordination conjugates with water and/or various solvents, in the various physical forms of the conjugates. Accordingly, the above formulae are to be understood to include and represent those various hydrates and/or solvates. It is also to be understood that the non-hydrates and/or non-solvates of the conjugate formulae are described by such formula, as well as the hydrates and/or solvates of the conjugate formulae.

The conjugates described herein can be expressed by the generalized descriptors B, L and D, for example B-L-D, where B is a cell surface receptor binding ligand (a.k.a. a “binding ligand”), L is a linker that may include one or more releasable portions (i.e. a releasable linker) and L may be described by, for example, one or more of the groups AA, L¹ or L² as defined herein, and D represents a drug covalently attached to the conjugates described herein.

The conjugates described herein can be described according to various embodiments including but not limited to
B-L²-AA-L²-L¹-D
B-L²-AA-L²-AA-L¹-D
B-L²-AA-L²-AA-L²-L¹-D
   B-AA-L¹-D
   B-(AA)₂-L¹-D
   B-(AA)₃-L¹-D
   B-(AA)₄-L¹-D
   B-(AA)₅-L¹-D
      wherein B, AA, L¹, L² and D are defined by the various embodiments described herein, or a pharmaceutically acceptable salt thereof.

As used herein, the term cell surface receptor binding ligand (aka a “binding ligand”), generally refers to compounds that bind to and/or target receptors that are found on cell surfaces, and in particular those that are found on, over-expressed by, and/or preferentially expressed on the surface of pathogenic cells. Illustrative ligands include, but are not limited to, vitamins and vitamin receptor binding compounds.

Illustrative vitamin moieties include carnitine, inositol, lipoic acid, pyridoxal, ascorbic acid, niacin, pantothenic acid, folic acid, riboflavin, thiamine, biotin, vitamin B₁₂, and the lipid soluble vitamins A, D, E and K. These vitamins, and their receptor-binding analogs and derivatives, constitute the targeting entity covalently attachment to the linker. Illustrative biotin analogs that bind to biotin receptors include, but are not limited to, biocytin, biotin sulfoxide, oxybiotin, and the like).

Illustrative folic acid analogs that bind to folate receptors include, but are not limited to folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. Other folates useful as complex forming ligands for this disclosure are the folate receptor-binding analogs aminopterin, amethopterin (also known as methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N-methylpteroylglutamic acid (dichloromethotrexate). The foregoing folic acid analogs and/or derivatives are conventionally termed “folates,” reflecting their ability to bind with folate-receptors, and such ligands when conjugated with exogenous molecules are effective to enhance transmembrane transport, such as via folate-mediated endocytosis as described herein.

In some embodiments, the binding ligand is folate or derivative thereof. In some embodiments, the binding ligand is of the formula

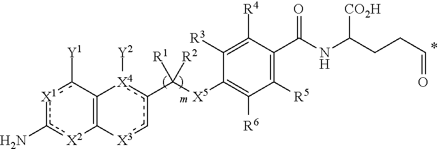

wherein

R¹ and R² in each instance are independently selected from the group consisting of H, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR, —SR⁸, —NR⁸R^{8′}, —C(O)R⁸, —C(O)OR⁸ or —C(O)NR⁸R^{8′};

R¹, R⁴, R⁵ and R⁶ are each independently selected from the group consisting of H, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, C₁-C₆ alkyl, C₂-C₆ alkenyl or C₂-C₆ alkynyl;

X¹ is —NR¹¹—, ═N—, —N═, —C(R¹¹)═ or —C(R¹¹)—;

X² is —NR^{11′} or ═N—;

X³ is —NR^{11′}—, —N═ or —C(R^{11′})═;

X⁴ is —N═ or —C═;

X⁵ is —NR¹²— or —CR¹²R^{12′}—;

Y¹ is H, —OR¹³, —SR¹³ or —NR¹³R^{13′} when X¹ is —N═ or —C(R¹¹)═, or Y¹ is ═O when X¹ is —NR¹¹—, ═N— or —C(R¹¹)—;

Y² is H, C₁-C₆ alkyl, C₂-C₆ alkenyl, —C(O)R¹⁴, —C(O)OR¹⁴, —C(O)NR¹⁴R^{14′} when X⁴ is —C═, or Y² is absent when X⁴ is —N═;

R¹¹, R^{11′}, R^{11″}, R¹², R^{12′}, R¹³, R^{13′}, R¹⁴ and R^{14′} are each independently selected from the group consisting of H, C₁-C₆ alkyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

wherein * is a covalent bond to the rest of the drug conjugate.

It will be appreciate that when B is described according to the formula

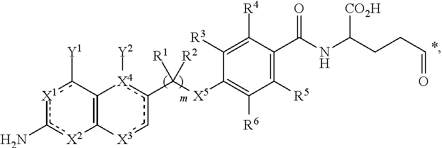

that both the D- and L-forms are contemplated. In some embodiments, B is of the formula

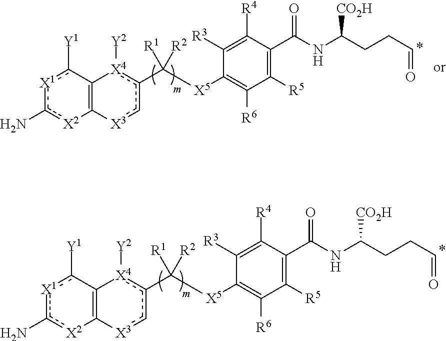

where each of R¹, R², R³, R⁴, R⁵, R⁶, Y¹, Y², X¹, X², X³, X⁴, X⁵, m and * are as defined herein.

In some embodiments described herein, R¹ and R² are H. In some embodiments described herein, m is 1. In some embodiments described herein, R³ is H. In some embodiments described herein, R⁴ is H. In some embodiments described herein, R⁵ is H. In some embodiments described herein, R⁶ is H. In some embodiments described herein, R³, R⁴, R⁵ and R⁶ are H. In some embodiments described herein, X¹ is —NR¹¹—, and R¹¹ is H. In some embodiments described herein, X² is ═N—. In some embodiments described herein, X³ is —N═. In some embodiments described herein, X⁴ is —N═. In some embodiments described herein, X³ is —NR¹¹—, and R¹¹ is H; X² is ═N—; X³ is —N═; and X⁴ is —N═. In some embodiments described herein, X⁵ is —NR¹²—, and R¹² is H. In some embodiments, Y¹ is ═O. In some embodiments, Y² is absent. In some embodiments, B is of the formula

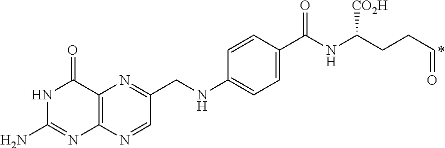 [SMILES: Nc1nc2ncc(CNc3ccc(C(=O)N[C@@H](CCC=O)C(=O)O)cc3)nc2c(=O)[nH]1]

wherein * is a covalent bond to the rest of the drug conjugate.

In some embodiments, B is of the formula

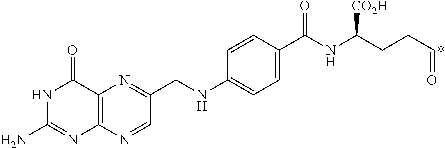 [SMILES: Nc1nc2ncc(CNc3ccc(C(=O)N[C@H](CCC=O)C(=O)O)cc3)nc2c(=O)[nH]1]

wherein * is a covalent bond to the rest of the drug conjugate.

It will be appreciated that in certain embodiments, the conjugates described herein can be represented by the exemplary formulae

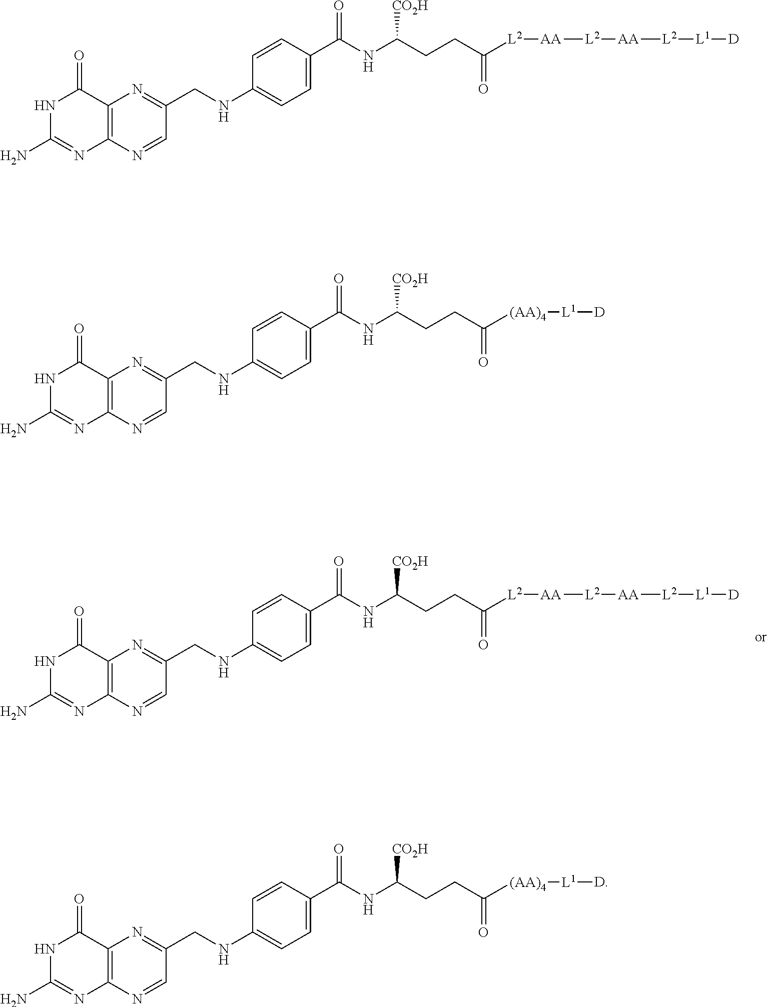

The linker for connected B and D in the conjugates described herein can be represented by the groups AA, L¹ and/or L².

AA is an amino acid as defined herein. In certain embodiments, AA is a naturally occurring amino acid. In certain embodiments, AA is in the L-form. In certain embodiments, AA is in the D-form. It will be appreciated that in certain embodiments, the conjugates described herein will comprise more than one amino acid as portions of the linker, and the amino acids can be the same or different, and can be selected from a group of amino acids. It will be appreciated that in certain embodiments, the conjugates described herein will comprise more than one amino acid as portions of the linker, and the amino acids can be the same or different, and can be selected from a group of amino acids in D- or L-form. In some embodiments, each AA is independently selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

In some embodiments, each AA is independently selected from the group consisting of L-asparagine, L-arginine, L-glycine, L-aspartic acid, L-glutamic acid, L-glutamine, L-cysteine, L-alanine, L-valine, L-leucine, L-isoleucine, D-asparagine, D-arginine, D-glycine, D-aspartic acid, D-glutamic acid, D-glutamine, D-cysteine, D-alanine, D-valine, D-leucine and D-isoleucine. In some embodiments, each AA is independently selected from the group consisting of Asp, Arg, Glu and Cys. In some embodiments, each AA is independently selected from the group consisting of Asp, Arg, Glu and D-Cys. In some embodiments, each AA is independently selected from the group consisting of Asp, Arg and Glu. In some embodiments, the sequence of AA in the linker is -Asp-Arg-Asp-Asp-.

It will be appreciated that conjugates described herein can contain a self-immolative linker portion. In some embodiments, this self-immolative linker portion, when present, can be referred to as a “releasable linker”. As used herein, the term “releasable linker” refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It is understood that a cleavable bond can connect two adjacent atoms within the releasable linker and/or connect other linkers or B and/or D, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, such as another linker, a drug or binding ligand, the releasable linker becomes separated from the other moiety following breaking of the bond.

The lability of the cleavable bond can be adjusted by, for example, substituents at or near the cleavable bond, such as including alpha-branching adjacent to a cleavable disulfide bond, increasing the hydrophobicity of substituents on silicon in a moiety having silicon-oxygen bond that may be hydrolyzed, homologating alkoxy groups that form part of a ketal or acetal that may be hydrolyzed, and the like.

In some embodiments, L¹ is a releasable linker. In some embodiments, L¹ is of the formula selected from the group consisting of

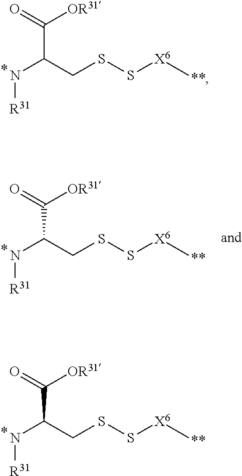

wherein

each of R³¹ and R^{31′} is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

X⁶ is independently a C₁-C₆ alkyl, C₂-C₆ heteroalkyl or C₆-C₁₀ aryl, wherein each hydrogen atom in C₁-C₆ alkyl, C₁-C₆ heteroalkyl and C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR⁴S(O)NR³⁵R^{35′}, —NR³⁴S(O)₂NR³⁵R^{35′}, —C(O)R³⁴, —C(O)OR³⁴ or —C(O)NR³⁴R^{34′};

each R³², R^{32′}, R³³, R^{33′}, R³⁴, R^{34′}, R³⁵ and R^{35′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

wherein ** is a covalent bond to a nitrogen atom of a tertiary amine on the tertiary amine containing drug; and * is a covalent bond to the rest of the drug conjugate.

In some embodiments, X⁶ is C₁-C₆ alkyl; wherein each hydrogen atom in C₁-C₆ alkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}. In some embodiments, X⁶ is methyl, ethyl, n-propyl, iso-propyl, n-butyl, sec-butyl or n-pentyl. In some embodiments, X⁶ is C₁-C₆ heteroalkyl; wherein each hydrogen atom in C₁-C₆ heteroalkyl is independently optionally substituted by halogen, C₁-C₆ alkyl, C₂-C₆ alkenyl, C₂-C₆ alkynyl, C₃-C₈ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR⁴S(O)NR³⁵R^{35′}, —NR³⁴S(O)₂NR³⁵R^{35′}, —C(O)R³⁴, —C(O)OR³⁴ or —C(O)NR³⁴R^{34′}. In some embodiments, X⁶ is C₁-C₆ heteroalkyl comprising one heteroatom selected from the group consisting of N, O and S.

In some embodiments, X⁶ is C₆-C₁₀ aryl, wherein each hydrogen atom in C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R³⁴. In some embodiments, X⁶ is phenyl.

In some embodiments, the releasable linker can be of the formula

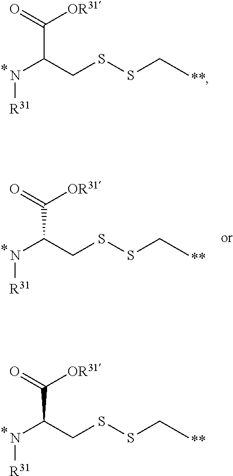

wherein ** is a covalent bond to a nitrogen atom of a tertiary amine on the tertiary amine containing drug; and * is a covalent bond to the rest of the drug conjugate.

In some embodiments, the releasable linker can be of the formula

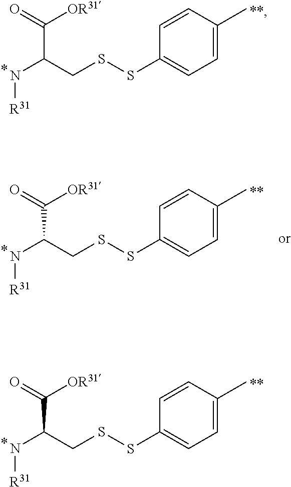

wherein ** is a covalent bond to a nitrogen atom of a tertiary amine on the tertiary amine containing drug; and * is a covalent bond to the rest of the drug conjugate.

L² can be present or absent in the linker portion of conjugates described herein. When L² is present, L² can be any group covalently attaching portions of the linker to the binding ligand, portions of the linker to one another, or to D. It will be understood that the structure of L² is not particularly limited in any way. It will be further understood that L² can comprise numerous functionalities well known in the art to covalently attach portions of the linker to the binding ligand, portions of the linker to one another, or to D, including but not limited to, alkyl groups, ether groups, amide groups, carboxy groups, sulfonate groups, alkenyl groups, alkynyl groups, cycloalkyl groups, aryl groups, heterocycloalkyl, heteroaryl groups, and the like. In some embodiments, L² is a linker of the formula

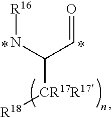

wherein

R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R², —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)R²¹, —NR²⁰S(O)NR²¹R^{21′}, —NR²⁰S(O)₂NR²¹R^{21′}, —C(O)R²², —C(O)OR²² or —C(O)NR²⁰R^{20′};

each R¹⁷ and R^{17′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R², —OC(O)NR²²R^{22′}, —OS(O)R²², —OS(O)₂R²², —SR², —S(O)R², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R², —NR²²S(O)₂R²³, —NR²S(O)NR²³R^{23′}, —NR²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵SR^{25′}, —C(O)R²⁴, —C(O)OR²⁴ or —C(O)NR²⁴R^{24′};

R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR^{2S}, —(CH₂)ₚ(OCH₂)_{q}OR²⁸, (CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

wherein each * is a covalent bond to the rest of the drug conjugate.

It will be appreciate that when L² is described according to the formula

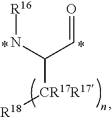

that both the R- and S-configurations are contemplated. In some embodiments, L² is of the formula
wherein each of R¹⁶, R¹⁷, R^{17′}, R¹⁸, n and * are as defined herein.

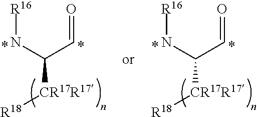

In some embodiments, R¹⁶ is H. In some embodiments, R¹⁸ is selected from the group consisting of H, 5- to 7-membered heteroaryl, —OR², —NR²C(O)R²⁷, —NR²C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR², —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

wherein each * is a covalent bond to the rest of the drug conjugate.

In some embodiments, R¹⁸ is selected from the group consisting of H, 5- to 7-membered heteroaryl, —OR²⁶, NR²⁶C(O)R²⁷, —NR²⁶C(O)NR²⁷R^{27′}, —NR²⁶C(═NR^{26″})NR²⁷R^{27′} and —C(O)NR²⁶R^{26′}, wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹, each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;

R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar); R²⁸ is H or sugar;

n is 1, 2, 3, 4 or 5;

p is 1, 2, 3, 4 or 5; and

q is, 2, 3, 4 or 5;

wherein * is a covalent bond to the rest of the drug conjugate.

In some embodiments, each L² is independently selected from the group consisting of

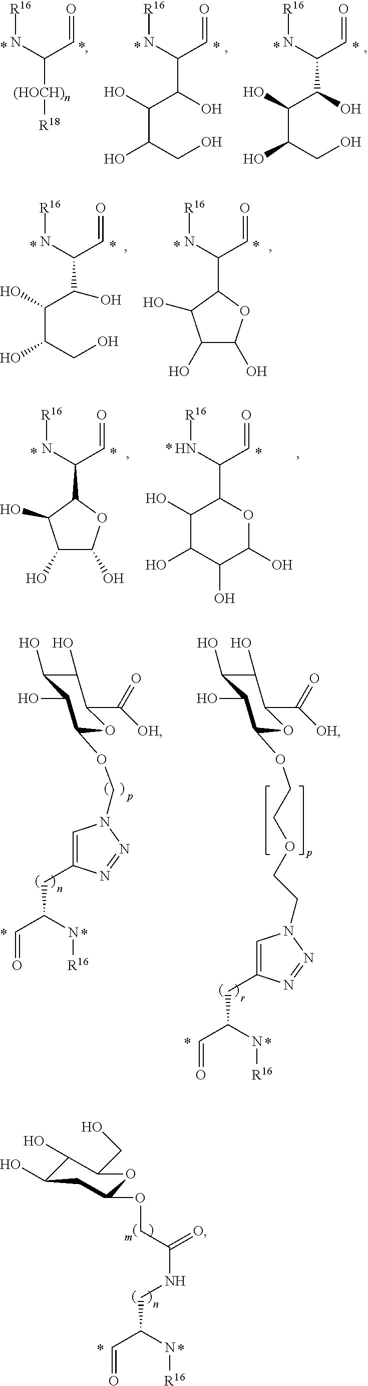

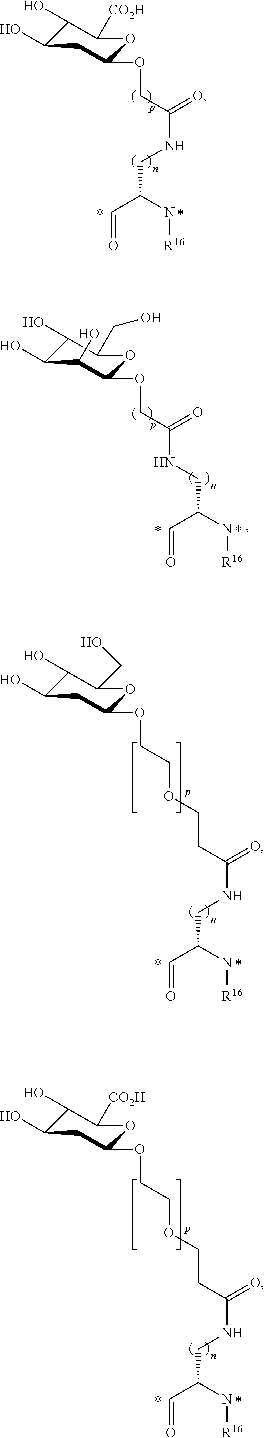

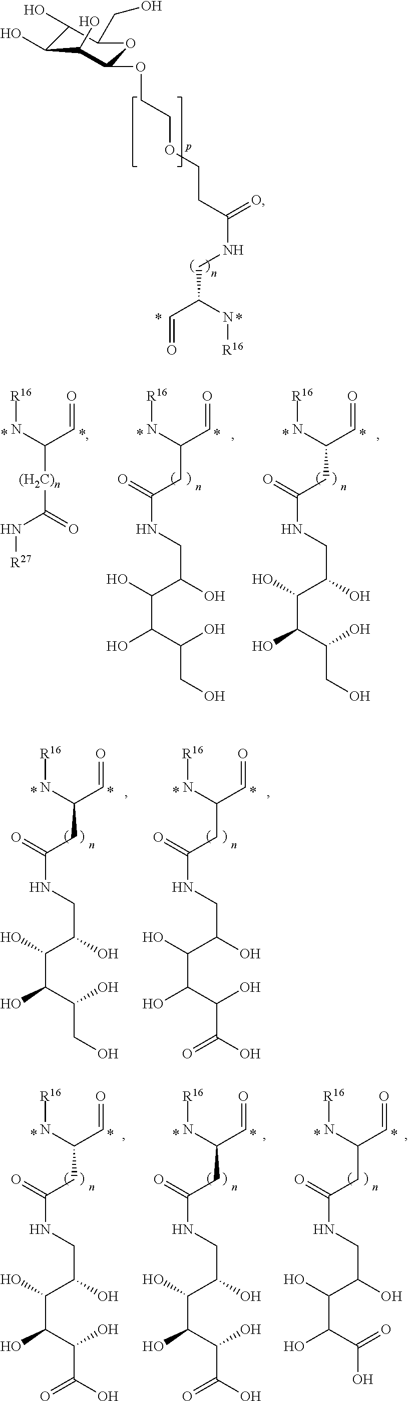

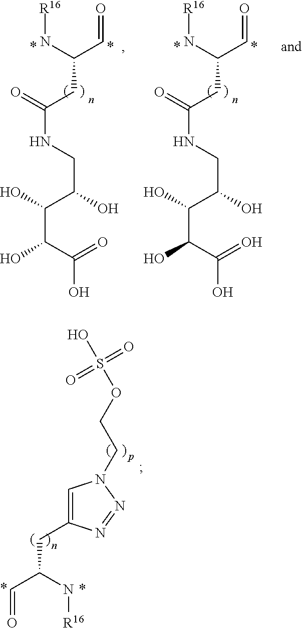

and combinations thereof,
wherein

R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each each R¹⁹, R^{19′}, R²⁰, R^{20′}, R²¹, R^{21′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar); R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

wherein each * is a covalent bond to the rest of the drug conjugate.

In some embodiments, L² is selected from the group consisting of

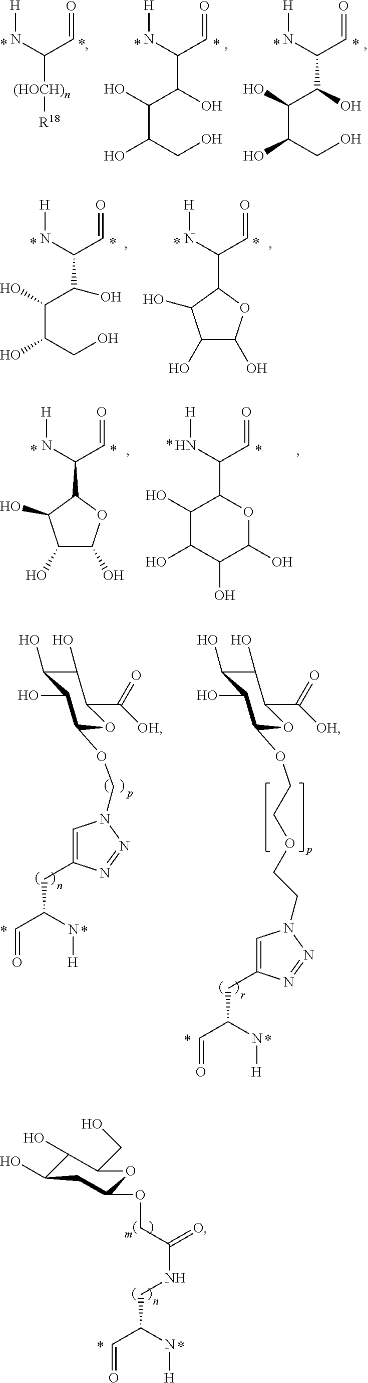

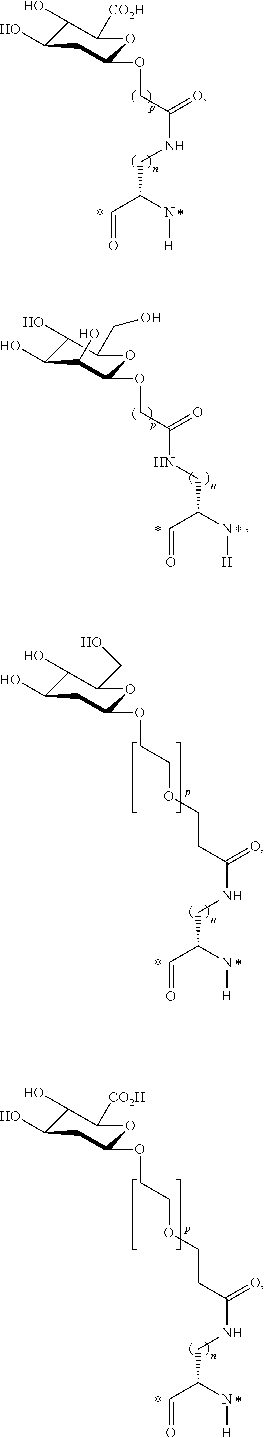

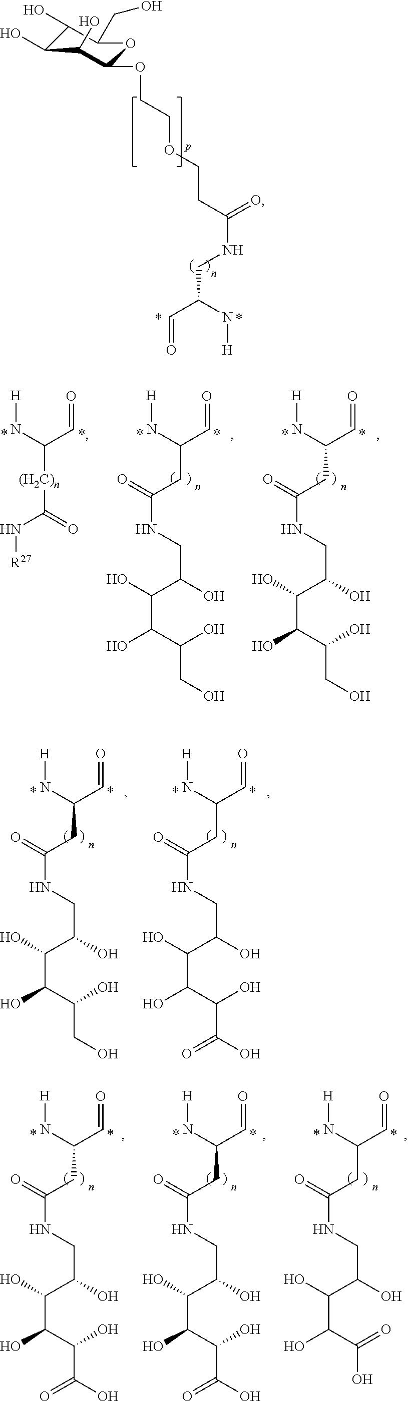

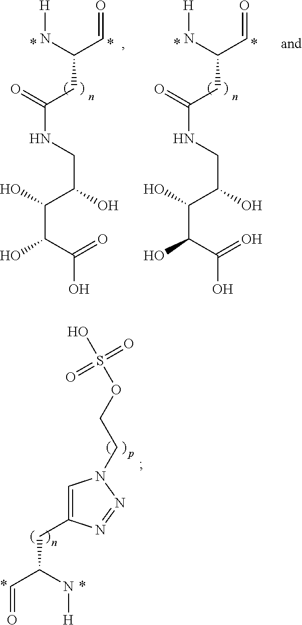

and combinations thereof,

wherein

R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR¹⁸, —(CH₂)ₚ(OCH₂)_{q}OR², —(CH₂)ₚ(OCH₂CH₂)_{q}OR², —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₈ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5; and
q is, 2, 3, 4 or 5;
wherein each * is a covalent bond to the rest of the drug conjugate.

The drug, D, for use in connection with the present disclosure can be any drug known to one of skill in the art that contains a tertiary amine functional group (also referred to herein as a “tertiary amine containing drug”). It will be appreciated that the identity of the drug for use in connection with the conjugates described herein is not particularly limited except that the drug contains a tertiary amine functional group. The tertiary amine containing drug may be any tertiary amine containing drug that induces a desired local or systemic effect. It is known that tertiary amines are an extremely important in various classes of compounds from drug discovery. It will be appreciated that such tertiary amine containing drug can be man-made or can be natural products. It will be appreciated that such tertiary amine containing drug can be man-made or can be natural products. Such drugs include broad classes of compounds. In general, this includes: analgesic agents; anesthetic agents; antiarthritic agents; respiratory drugs, including antiasthmatic agents; anticancer agents, including antineoplastic agents; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, CNS agents; beta-blockers and antiarrhythmic agents; central nervous system stimulants; serotonin agents (enhancers, transport or re-uptake inhibitors); alpha adrenergic antagonists or agonists; cough and cold preparations, including decongestants; antitussives; diuretics; genetic materials; gastrointestinal (GI) motility agents; herbal remedies; hormones; hormonolytics; hypnotics; hypoglycemic agents; immunosuppressive agents; leukotriene inhibitors; mitotic inhibitors; muscle relaxants; narcotic antagonists; opiod modulators; nicotine; nictone/acetylcholine antagonists or agonists; nutritional agents, such as vitamins, essential amino acids and fatty acids; ophthalmic drugs such as antiglaucoma agents; parasympatholytics; peptide drugs; psychostimulants; sedatives; steroids; sympathomimetics; tranquilizers; and vasodilators including general coronary, peripheral and cerebral.

Examples of tertiary amine-containing antibiotic drugs include clindamycin, ofloxacin/levofloxacin, pefloxacin, quinupristine, rolitetracycline, and cefotiam.

Examples of tertiary amine-containing antifungal drugs include butenafine, naftifine, and terbinafine.

Examples of tertiary amine-containing antimalarials and antiprotozoals drugs include amodiaquine, quinacrine, sitamaquine, quinine.

Examples of tertiary amine-containing HIV protease inhibitor drugs include saquinavir, indinavir, atazanavir and nelfinavir. Anti-HIV drugs also include maraviroc and aplaviroc for inhibition of HIV entry.

Examples of tertiary amine-containing anticonvulsants/antispasmodics drugs from include atropine, darifenancin; dicyclomine; hyoscayamine, tiagabine, flavoxate; and alverine.

Examples of tertiary-amine containing antidepressant drugs include amitriptyline, adinazolam, citalopram, cotinine, clomipramine, doxepin, escitalopram, femoxetine, imipramine, minaprine, moclobemide, mianserin, mirtazapine, nefazodone, nefopam, pipofenazine, promazine, ritanserin, trazodone, trimipramine and venlafaxine.

Examples of tertiary amine-containing antiemetic drugs include aprepitant, buclizine, cilansetron, cyclizine, dolasetron, granisetron, meclizine, ondansetron, palonosetron, ramosetron, thiethylperazine, trimethobenzamide, scopolamine, and prochlorperazine.

Examples of tertiary amine-containing antihistamine drugs include acetprometazine, azatadine, azelastine, brompheniramine, carbinoxamine, chlorpheniramine, clemastine, dexobrompheniramine, diphenhydramine, diphenylpyraline, doxepin, emadastine, loratadine, mequitazine, olopatadine, phenindamine, pheniramine, promethazine, tripelennamine, triprolidine, astemizole, cetirizine, fexofenadine, terfenadine, latrepirdine, ketotifen, cyproheptadine, hydroxyzine, clobenzepam doxylamine, cinnarizine, orphenadrine.

Examples of tertiary amine-containing antiparkinsonian drugs include cabergoline, ethopropazine, pergolide, selegiline, metixene, biperiden, cycrimine, procycladine and apomorphine.

Examples of tertiary amine-containing antipsychotic drugs include acetophenazine, amisulpride, aripiprazole, bifeprunox, blonanserin, cariprazine, carphenazine, clopenthixol, clozapine, dehydro aripiprazole, someperidone, droperidol, flupenthixol, fluphenazine, fluspirilene, haloperidol, iloperidone, lurasidone, mesoridazine, molindole, nemanopride, olanzapine, perospirone, perphenazine, PF— 0021 7830 (Pfizer), pipotiazine, propericiazine, quetiapine, remoxipride, risperidone, sertindole, SLV-31 3 (Solvay/Wyeth), sulpiride, thioproperazine, thioridazine, thiothixene, trifluoperazine, ziprasidone, zotepine, pimozide, benzquinamide, triflupromazine, tetrabenazine, melperon, asenapine, chlorprothixene, spiperone and chlorpromazine.

Examples of tertiary amine-containing anxiolytic drugs include buspirone, and loxapine.

Examples of tertiary amine-containing nootroopic (memory and cognitive enhancers) drugs include donepezil, galantamine, latrepirdine, nicotine, TC-5616 (Targacept, Inc.) having the IUPAC name: N-[(2S,3S)-2-(pyridin-3-ylmethyl)-1-azabicyclo[2.2.2]oct-3-yl]-1-benzofuran-2-carboxamide.

Examples of tertiary amine-containing drugs for erectile dysfunction include apomorphine and sildenafil.

Examples of tertiary amine-containing drugs for migraine headache include almotriptan, naratriptan, rizatriptan, sumatriptan, zolmitriptan, dihydroergotamine, ergotamine, eletripan and iisuride.

Examples of tertiary amine-containing drugs for the treatment of alcoholism include naloxone and naltrexone. Other narcotic antagonist amine containing drugs for treatment of substance abuse include levallorphan, nalbuphine, nalorphine and nalmefene.

Examples of a tertiary amine-containing drug for the treatment of addiction include buprenorphine, isomethadone, levomethadyl acetate, methadyl acetate, nor-acetyl levomethadol, and normethadone.

Examples of tertiary amine-containing muscle relaxant drugs include cyclobenzaprine, nefopam, tolperisone, orphenadrine, and quinine.

Examples of tertiary amine-containing nonsteroidal anti-inflammatory drugs include etodolac, meloxicam, ketorolac, lornoxicam and tenoxicam. Examples of tertiary amine-containing opioid drugs include alfentanil, anileridine, buprenorphine, butorphanol, clonitazene, codeine, dihydrocodeine, dihydromorphin, fentanyl, hydromorphone, meperidine, metazocine, methadone, morphine, oxycodone, hyrdocodone, oxymorphone, pentazocine, remifentanil, and sufentanil.

Examples of other tertiary amine-containing analgesic drugs include methotrimeprazine, tramadol, nefopam, phenazocine, propiram, quinurjramine, thebaine and propoxyphene.

Examples of tertiary amine-containing sedatives/hypnotics include eszopiclone, flurazepam, propiomazine, and zopiclone.

Examples of tertiary amine-containing local analgesic drugs include bupivacaine, dexmedetomidine, dibucaine, dyclonine, lodicaine, mepivacaine, procaine, and tapentadol and ropivacaine.

Examples of tertiary amine-containing antianginals include ranozaline, bepridil.

Examples of tertiary amine-containing antiarrhythmics include amiodarone, aprindine, encainide, moricizine, procainamide, diltiazem, verapamil, bepridil.

Examples of tertiary amine-containing antihypertensives include azelnidipine, deserpidine, ketanserin, reserpine, and sildenafil.

Examples of tertiary amine-containing antithrombotics include clopidogrel and ticlopidine.

Examples of tertiary amine-containing antineoplastic drugs include dasatinib, flavopiridol, gefitinib, imatinib, sunitinib, topotecan, vinblastine, vincristine, fincesine, vinorelbine, vinorelbine, tamoxifen, tremifene, and tesmilifene. Examples of tertiary amine-containing drugs parent drugs for use in treating irritable bowel syndrome (IBS) from which the prodrugs of the invention are derived include asimadoline.

Examples of other tertiary amine-containing drugs useful in connection with the present disclosure include antimuscarinics and anticholinergics such as benzotropine, procyclidine and trihexylphenidyl; alpha andrenergic blockers such as dapiprazole, dexmedetomidine and nicergoline; anorexics such as diethylpropian, benzapehtamine, phendimetrazine, and sibutramine; antidiarrhels such as diphenoxylate and loperamide, antikinetic and antihypertensives such as clonidine; antiosteoporotics such as raloxifene; antipruritics such as methyldilazine; antitussives such as dextromethorphan; antiulceratives such as pirenzepine; cholinesterase inhibitors such as galantamine; gastroprokinetics such as alvimopan, cisapride, and piboserod; miglustat for treating glycosphingolipid lysosomal storage disorder; clomifene as gonad stimulating prinicipal; neuromuscular blockers such as dihydro-beta-erythrodoidine, niotropics such as rivastigmine, oxytocics such as methylergonovine; antiametics such as chloroquine; respiratory stimulants such as doxapram; muscarinic receptor antagonists for treating urinary incontinence such as oxybutynin and solifenacin; calcium channel blockers such as flunarizine; anthelmintics such as diethylcarbamazine and quinacrine; miotics such as physostigmine; neuroprotectives such as lubeluzole; immunosuppressants such as mycophenolate mofetil; and stimulants such as nicotine.

Examples of suitable tertiary amine containing drugs for use in connection with the present teachings, include but are not limited to, morphine, hydrocodone, oxycodone, codeine, mitragynol, vinblastine, vincristine, vindesine, vinorelbine, clindamycin, novobiocin, retapamulin, dimethylpipBOR, N,N-dimethylsitafloxacin, rifampin, azithromycin, venlafaxine, mirtazapine, escitalopram, porfiromycin, pamamycin 601, macromerine, tatreponerine 8, imatinib, aripiprazole, buprenorphine, sildenafil, quetiapine, methylphenidate, doxycycline, solifenacin, lidocaine, eszopiclone, tamoxifen, beloranib, diphenylhydramine and tubulysin.

The conjugates described herein can be used for both human clinical medicine and veterinary applications. Thus, the host animal harboring the population of pathogenic cells and treated with the conjugates described herein can be human or, in the case of veterinary applications, can be a laboratory, agricultural, domestic, or wild animal. The conjugates described hereincan be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The conjugate, compositions, methods, and uses described herein are useful for treating diseases caused at least in part by populations of pathogenic cells, which may cause a variety of pathologies in host animals. As used herein, the term “pathogenic cells” or “population of pathogenic cells” generally refers to cancer cells, infectious agents such as bacteria and viruses, bacteria- or virus-infected cells, inflammatory cells, activated macrophages capable of causing a disease state, and any other type of pathogenic cells that uniquely express, preferentially express, or overexpress cell surface receptors or cell surface antigens that may be bound by or targeted by the conjugates described herein. Pathogenic cells can also include any cells causing a disease state for which treatment with the conjugates described herein results in reduction of the symptoms of the disease. For example, the pathogenic cells can be host cells that are pathogenic under some circumstances such as cells of the immune system that are responsible for graft versus host disease, but not pathogenic under other circumstances.

Thus, the population of pathogenic cells can be a cancer cell population that is tumorigenic, including benign tumors and malignant tumors, or it can be non-tumorigenic. The cancer cell population can arise spontaneously or by such processes as mutations present in the germline of the host animal or somatic mutations, or it can be chemically-, virally-, or radiation-induced. The conjugates described herein can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

The disclosure includes all pharmaceutically acceptable isotopically-labelled conjugates, and their Drug(s) incorporated therein, wherein one or more atoms are replaced by atoms having the same atomic number, but an atomic mass or mass number different from the atomic mass or mass number which predominates in nature.

Examples of isotopes suitable for inclusion in the conjugates, and their Drug(s) incorporated therein, include isotopes of hydrogen, such as ²H and ³H, carbon, such as ¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled conjugates, and their Drug(s) incorporated therein, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, and ¹³N, can be useful in Positron Emission Topography (PET) studies for examining substrate receptor occupancy. Isotopically-labeled conjugates, and their Drug(s) incorporated therein, can generally be prepared by conventional techniques known to those skilled in the art or by processes analogous to those described in the accompanying Examples using an appropriate isotopically-labeled reagents in place of the non-labeled reagent previously employed.

The conjugates and compositions described herein may be administered orally. Oral administration may involve swallowing, so that the conjugate or composition enters the gastrointestinal tract, or buccal or sublingual administration may be employed by which the conjugate or composition enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The conjugates and compositions described herein may also be used in fast-dissolving, fast-disintegrating dosage forms such as those described in Expert Opinion in Therapeutic Patents, 11 (6), 981-986, by Liang and Chen (2001). For tablet dosage forms, depending on dose, the conjugate may make up from 1 weight % to 80 weight % of the dosage form, more typically from 5 weight % to 60 weight % of the dosage form. In addition to the conjugates and compositions described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a conjugate as described herein, a film-forming polymer, a binder, a solvent, a humectant, a plasticizer, a stabilizer or emulsifier, a viscosity-modifying agent and a solvent.

Some components of the formulation may perform more than one function. Solid formulations for oral administration may be formulated to be immediate and/or modified release. Modified release formulations include delayed-, sustained-, pulsed-, controlled-, targeted and programmed release. Suitable modified release formulations for the purposes of the disaclosure are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The conjugates described herein can also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous.

Suitable devices for parenteral administration include needle (including micro-needle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of conjugates described herein used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus conjugates described herein can be formulated as a solid, semi-solid, or thixotropic liquid for administration as an implanted depot providing modified release of the active compound. Examples of such formulations include drug-coated stents and poly(lactic-coglycolic)acid (PGLA) microspheres. The conjugates described herein can also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm Sci, 88 (10), 955-958 by Finnin and Morgan (October 1999). Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release. The conjugates described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. The pressurized container, pump, spray, atomizer, or nebulizer contains a solution or suspension of the conjugates(s) of the present disclosure comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the conjugate is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules (made, for example, from gelatin or hydroxypropylmethylcellulose), blisters and cartridges for use in an inhaler or insufflator may be formulated to contain a powder mix of the conjugate described herein, a suitable powder base such as lactose or starch and a performance modifier such as Iso-leucine, mannitol, or magnesium stearate.

The lactose may be anhydrous or in the form of the monohydrate, preferably the latter. Other suitable excipients include dextran, glucose, maltose, sorbitol, xylitol, fructose, sucrose and trehalose. A typical formulation may comprise a conjugate of the present disclosure, propylene glycol, sterile water, ethanol and sodium chloride. Alternative solvents which may be used instead of propylene glycol include glycerol and polyethylene glycol.

The conjugates described here can be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present disclosure that two or more pharmaceutical compositions, at least one of which contains a conjugate as described herein, may conveniently be combined in the form of a kit suitable for co-administration of the compositions. Thus the kit of the present disclosure comprises two or more separate pharmaceutical compositions, at least one of which contains a conjugate as described herein, and means for separately retaining said compositions, such as a container, divided bottle, or divided foil packet. An example of such a kit is the familiar blister pack used for the packaging of tablets, capsules and the like. The kit of the present disclosure is particularly suitable for administering different dosage forms, for example parenteral, for administering the separate compositions at different dosage intervals, or for titrating the separate compositions against one another. To assist compliance, the kit typically comprises directions for administration and may be provided with a so-called memory aid.

### EXAMPLES

### Chemical Examples

### Materials

### Preparation of (4-(bromomethyl)phenyl)(tert-butyl)sulfane

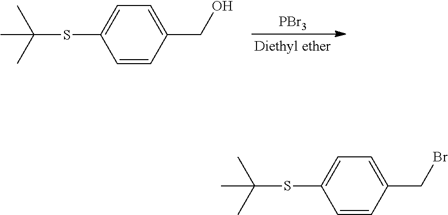

The starting material, [4-(tert-butylsulfanyl)phenyl]methanol (179 mg, 0.912 mmol, 1 eq.) was dissolved in diethyl ether (˜2 mL) and the solution cooled to 0° C. (ice-bath). Phosphorous tribromide (182 μL, 1.00 mmol, 1.1 eq.) was added drop-wise and the reaction mixture stirred for 30 minutes. The ice-bath was then removed and the reaction mixture stirred at room temperature for 1 hour. TLC and UPLC (10-100% ACN/50 mM pH7 NH₄HCO₃ buffer) showed the reaction went to completion. Excess phosphorous tribromide was quenched with ice and the reaction mixture worked up between water and diethyl ether. The aqueous layer was re-extracted with additional diethyl ether (2×10 mL). The combined organic layers were dried (Na₂SO₄), filtered and the volatiles evaporated in vacuo. The residue was purified on a 4 g silica column using 0-30% EtOAc/Petroleum ether to yield the brominated product as a white powder (202 mg, 86%).

¹H NMR (500 MHz, CD₂Cl₂): H=7.51 (d, J=11 Hz, 2H), 7.38 (d, J=8.5 Hz, 2H), 4.53 (s, 2H), 1.29 (s, 9H).

### Preparation of Compound 1-c

Compound 1-c was prepared according to the procedures described in U.S. Pat. No. 8,889,880, incorporated herein by reference for the preparation of compound 1-c, described therein as compound (7).

### Preparation of Compound 2-a

Compound 2-a was prepared according to the procedures described in Peltier, et al. “The Total Synthesis of Tubulysin D” J. Am. Chem. Soc. 128, 16018-19 (2006), incorporated herein by reference for the preparation of compound 2-a, described therein as compound (11).

### General Method for the Preparation of Compounds of the Type 5

The compounds of the type 5 can be prepared according to the methods described in U.S. Pat. No. 7,601,332, incorporated herein by reference for the disclosure of the methods for preparing compounds of the type 5.

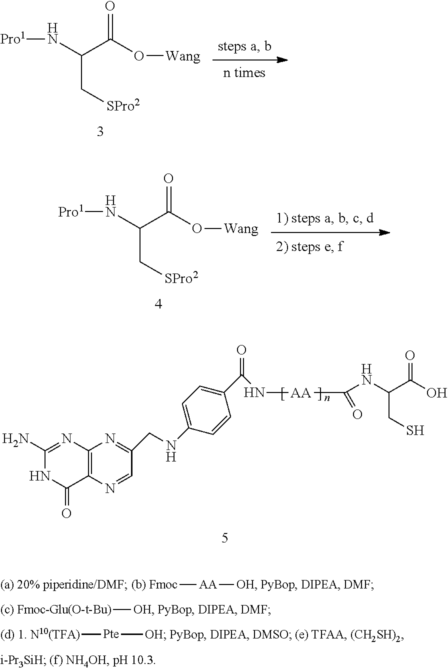

The folate-containing peptidyl fragment Pte-Asp-(AA)ₙ-Cys-OH (5) is prepared by a polymer-supported sequential approach using the Fmoc-strategy on an acid-sensitive Fmoc-Cys(Trt)-Wang resin (3). Pro¹ is Fmoc, Pro² is Trityl, and DIPEA is diisopropylethylamine. PyBop is applied as the activating reagent to ensure efficient coupling. Fmoc protecting groups are removed after each coupling step under standard conditions. Appropriately protected commercially available amino acid building blocks, such as Fmoc-Asp-OtBu (Sigma-Aldrich), Fmoc-Arg (Sigma-Aldrich), and the like, are used, and represented by in step (b) by Fmoc-AA-OH. Thus, AA refers to any amino acid starting material that is appropriatedly protected. The coupling sequence (steps (a) & (b)) involving Fmoc-AA-OH is performed “n” times to prepare solid-supported peptide (4), where n is an integer and may equal 0 to about 100. Following the last coupling step, the remaining Fmoc group is removed, and the peptide is sequentially coupled to a glutamate derivative (step (c)), deprotected, and coupled to TFA-protected pteroic acid (step (d)). Subsequently, the peptide is cleaved from the polymeric support upon treatment with trifluoroacetic acid, ethanedithiol, and triisopropylsilane (step (e)). These reaction conditions result in the simultaneous removal of the t-Bu, t-Boc, and Trt protecting groups. The TFA protecting group is removed upon treatment with base (step (f)) to provide the folate-containing Cys-containing peptidyl fragment (5).

### Preparation of Compound 5-1

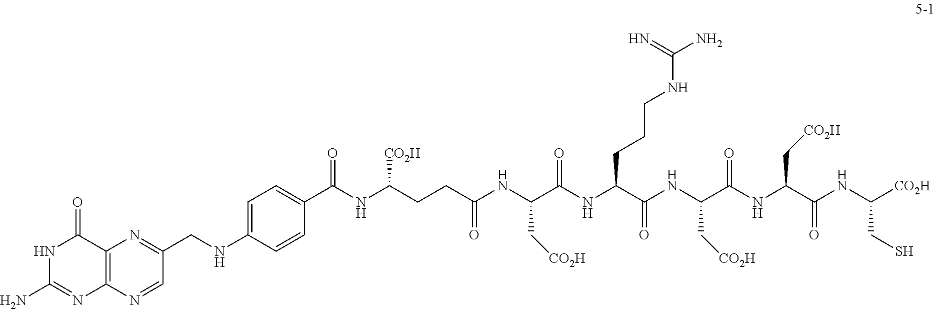 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

Compound 5-1 was prepared according to the general method described above using Fmoc-Asp-OtBu (Sigma-Aldrich) and Fmoc-Arg (Sigma-Aldrich) as the amino acid reagents in step b.

### Example 1: Synthesis of Compound 1

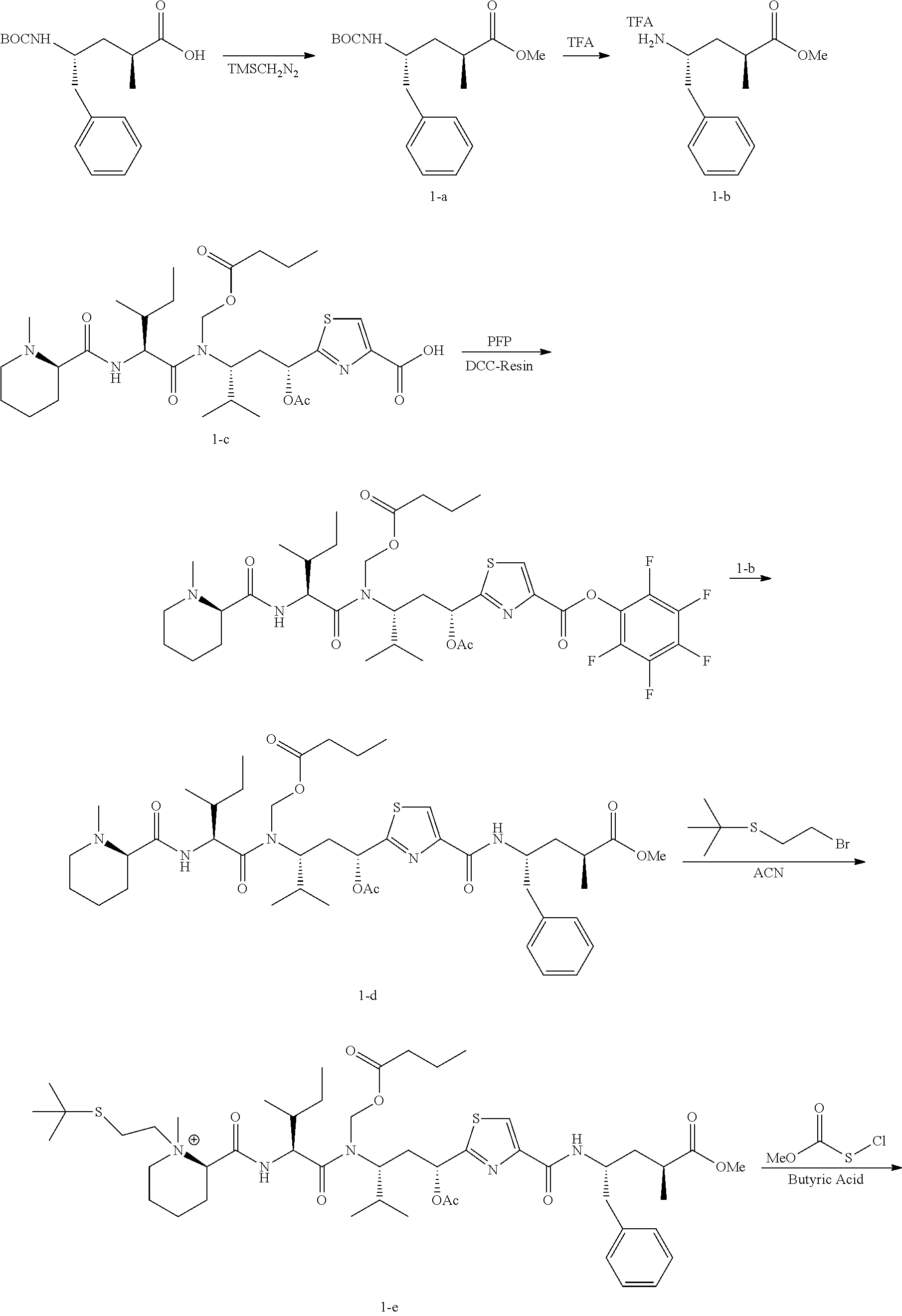

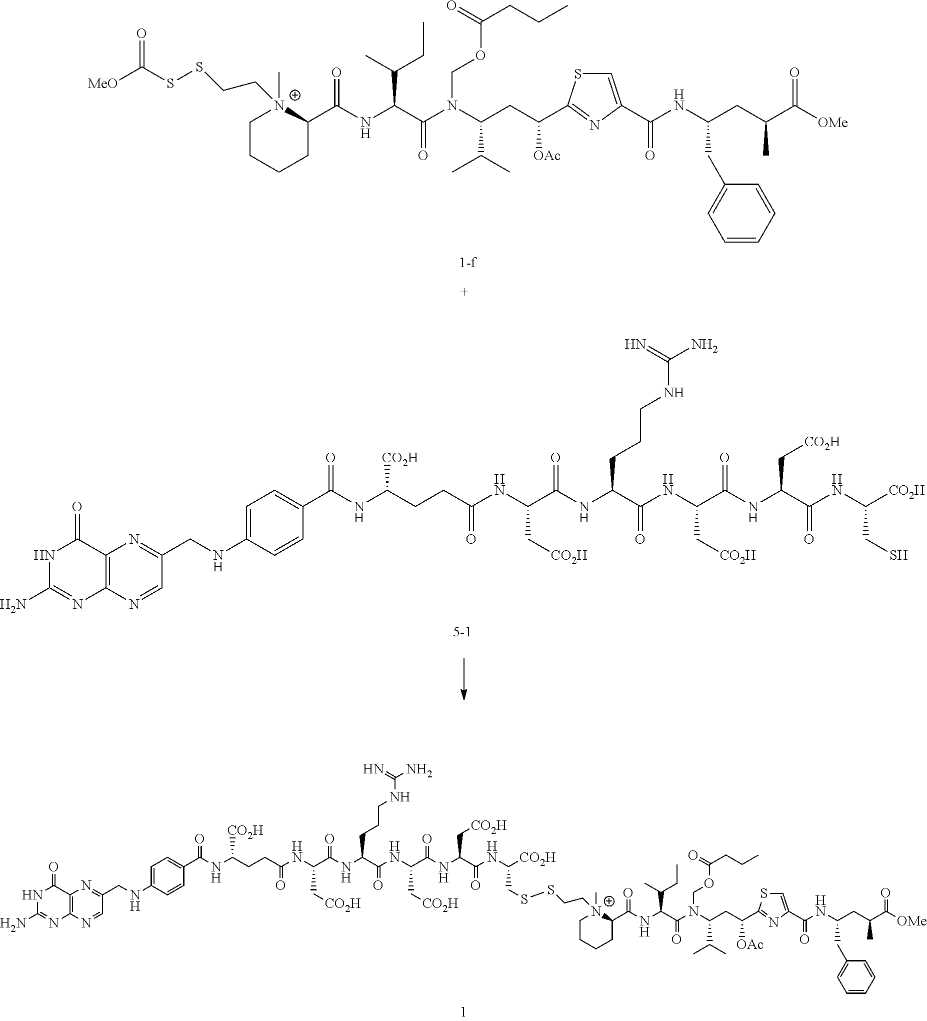

### Step 1 and 2: Synthesis of Methyl 1-a and 1-b

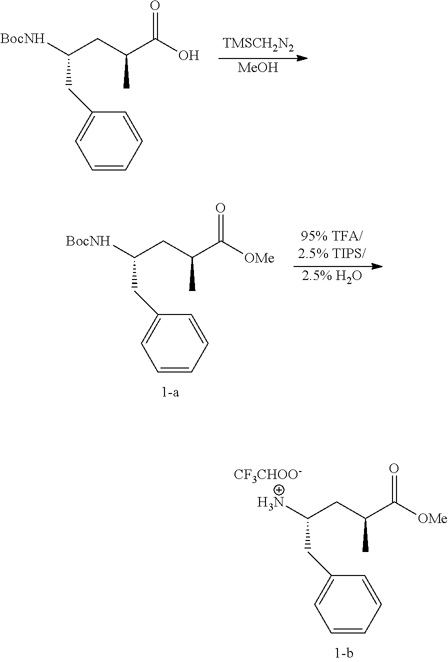 [SMILES: "MeOH" ; C[C@@H](C[C@H](Cc1ccccc1)NC(=O)OC(C)(C)C)C(=O)O ; COC(=O)[C@@H](C)C[C@H](Cc1ccccc1)NC(=O)OC(C)(C)C ; "2.5% H2O" ; "CF3CHOO-" ; COC(=O)[C@@H](C)C[C@@H]([NH3+])Cc1ccccc1]

Boc-protected Tup (200 mg, 0.65 mmol, 1 eq.) was dissolved in methanol (2.0 mL) and the solution cooled to 0° C. in an ice bath. TMS-diazomethane (0.65 mL, 2M solution in ether, 1.30 mmol, 2 eq.) was added drop-wise and the reaction was observed to turn from yellow to clear. Reaction progress was monitored by UPLC (10-100% ACN/50 mM pH7 NH₄HCO₃ buffer). A further 0.90 mL (1.80 mmol, 3 eq.) of TMS-diazomethane was required to effect completion. Acetic acid (0.1 mL) was used to quench excess, unreacted methylating agent and the solvent evaporated in vacuo to give 333 mg crude material.

Crude 1-a (13 mg, 0.040 mmol) was treated with 95% TFA/2.5% TIPS/2.5% H₂O. After 10 minutes, UPLC (10-100% ACN/50 mM pH7 NH₄HCO₃ buffer) showed complete Boc deprotection of the starting material. The cleavage solution was evaporated in vacuo and the resulting residue used without further purification.

LC/MS (ESI-QMS): 221.9 m/z=(M+H). R_{f}(10% MeOH/DCM) 0.56. ¹H NMR (500 MHz, MeOD): H=7.36-7.37 (m, 2H), 7.25-7.30 (m, 3H), 3.63 (s, 3H), 3.50-3.55 (m, 1H), 2.97 (dd, J=6.5 Hz, 6.5 Hz, 1H), 2.88 (dd, J=8 Hz, 8 Hz, 1H), 2.67-2.71 (m, 1H), 1.97-2.02 (1H, m), 1.60-1.66 (m, 1H), 1.15 (d, J=6.5 Hz, 3H).

### Step 3 and 4: Synthesis of 1-d

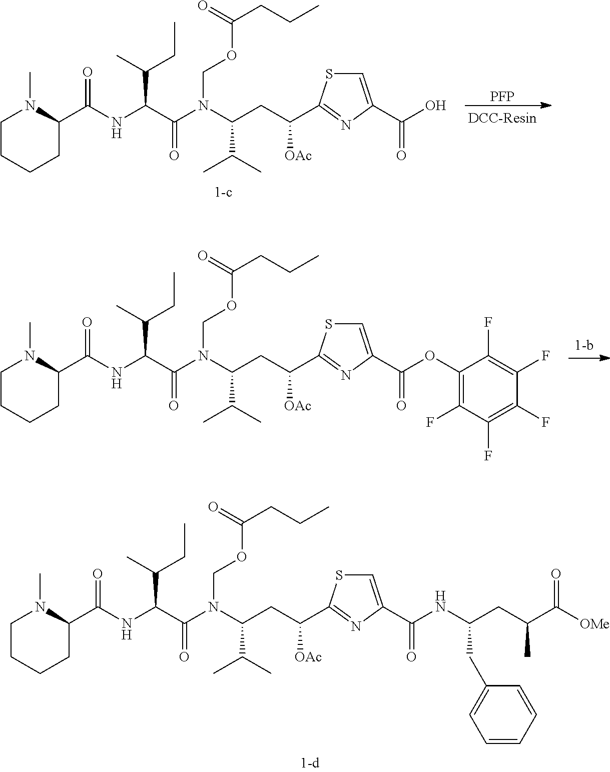

The tripeptide, 1-c (29 mg, 0.046 mmol, 1 eq.) and benzotriazol-1-yl-oxytripyrrolidinophosphonium hexafluorophosphate (PyBOP) (36 mg, 0.070 mmol, 1.5 eq.) were dissolved in NMP (1 ml) to which 1-b (10 mg, 0.046 mmol, 1 eq.) and triethylamine (129 μL, 0.928 mmol, 20 eq.) were added. Reaction progress was monitored by UPLC (10-100% ACN/50 mM pH7 NH₄HCO₃ buffer). A further 32 μL (5 eq.) triethylamine was needed to effect full conversion of the activated acid to the desired product. The reaction mixture was purified by reverse phase chromatography on a 12 g, C18 column using a 0-100% ACN/H₂O gradient. The fractions containing the product were collected and the acetonitrile removed under reduced pressure. The remaining aqueous layer was re-extracted with EtOAc (2×10 mL) and the organic extracts dried (Na₂SO₄), filtered and the volatiles evaporated in vacuo. After drying, 1-d was collected (6.2 mg, 16%).

LC/MS (ESI-QMS): 828.3. m/z=(M+H).). R_{f}(10% MeOH/DCM) 0.58. ¹H NMR (500 MHz, MeOD): H=8.09 (s, 1H), 7.23-7.25 (m, 4H), 7.16-7.18 (m, 1H), 6.15 (d, J=12 Hz, 1H), 5.87 (dd, J=12 Hz, 12 Hz, 1H), 5.52 (d, J=12 Hz, 1H), 4.63 (d, J=9 Hz, 1H), 4.31-4.36 (m, 1H), 3.60 (s, 3H), 2.93-2.94 (m, 1H), 2.89 (t, J=7.0 Hz), 2.58-2.62 (m, 2H), 2.46-2.50 (m, 1H), 2.19-2.30 (m, 3H), 2.18 (s, 3H), 2.15 (s, 3H), 2.06-2.11 (m, 1H), 1.96-2.01 (m, 2H), 1.57-1.77 (m, 7H), 1.51-1.56 (q, J=7.0 Hz, 3H), 1.14 (d, J=6.5 Hz, 3H), 1.06 (d, J=6.0 Hz, 3H), 0.97 (d, J=6.5 Hz, 3H), 0.95 (m, 1H), 0.91 (t, J=7.5 Hz, 4H), 0.85 (t, J=7.5 Hz, 3H), 0.80 (d, J=6.5 Hz, 3H).

### Step 5: Synthesis of 1-e

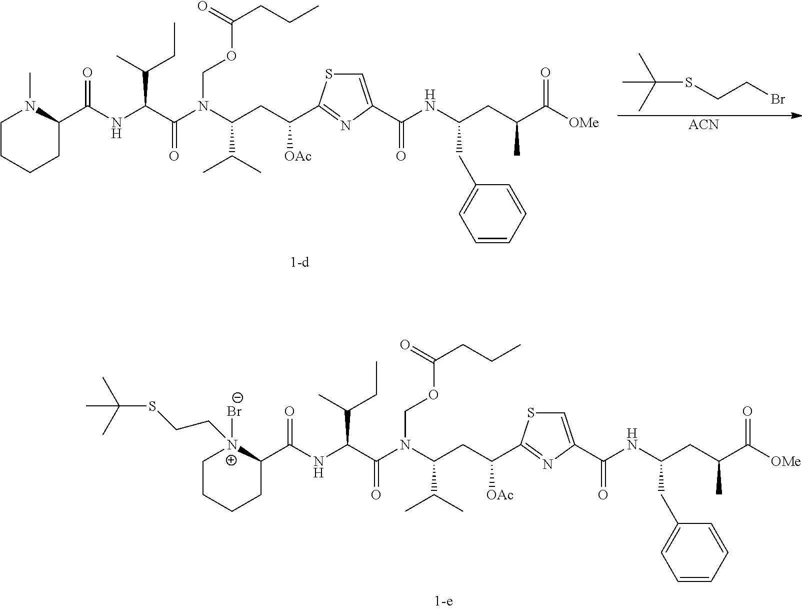

1-d (30 mg, 0.036 mmol, 1 eq.) was dissolved in acetonitrile (˜1 ml) to which 2-[(2-bromoethyl)sulfanyl]-2-methylpropane (36 mg, 0.18 mmol, 5 eq.) was added. The reaction was stirred under an argon atmosphere for 3 nights. TLC (10% MeOH/DCM) showed two spots corresponding to the starting material and desired product. The solvent was removed under reduced pressure and the remaining residue purified on a 4 g silica column using 100% EtOAc followed by 10% MeOH/DCM as the eluent. Fractions containing the product were collected and after removing the solvent under reduced pressure yielded 1-e (15 mg, 44%).

LC/MS (ESI-QMS): 944.6 m/z=(M+H). R_{f}(10% MeOH/DCM) 0.45. ¹H NMR (500 MHz, MeOD): H=8.73 (d, J=7 Hz, 1H), 8.10 (s, 1H), 7.95 (d, J=9 Hz, 1H), 7.18-7.26 (m, 4H), 7.15-7.18 (m, 1H), 5.94 (d, J=12 Hz, 1H), 5.87 (dd, J=12 Hz, 11 Hz, 1H), 5.54 (d, J=13 Hz, 1H), 4.59-4.65 (m, 1H, 4.18 (t, J=5.5 Hz, 1H), 3.91 (s, 1H), 3.60 (s, 3H), 3.21-3.24 (m, 4H), 2.87-2.91 (m, 4H), 1.89-2.02 (m, 8H), 1.54-1.74 (m, 7H), 1.36 (s, 9H), 1.34 (s, 3H), 1.15 (d, J=6.5 Hz, 3H), 1.07 (d, J=6.5 Hz, 3H), 1.02 (m, 1H), 1.01 (d, J=6.5 Hz, 3H), 0.94 (t, J=7.5 Hz, 4H), 0.86-0.89 (m, 6H).

### Step 6 and 7: Synthesis of 1-f and Compound 1

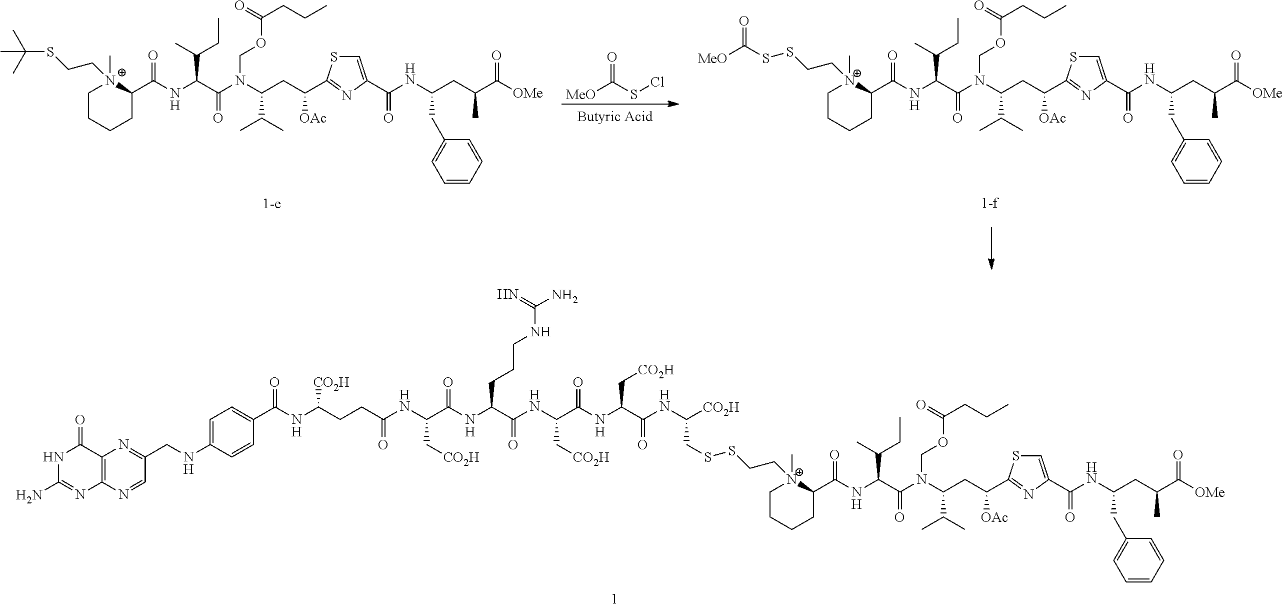

4.5 mg of 1-e (4.8×10³ mmol) was dissolved in 5.3 μL of methoxycarbonylsulfenyl chloride (0.059 mmol, 12 eq.) and 130 ILL butyric acid with 200 μL dichloromethane. After 5 minutes, LC/MS showed the appearance of 1-f. After the starting material was consumed, dichloromethane was removed with the rotary evaporator and the resulting residue placed under high vacuum until dry. The residue was dissolved in 0.5 mL of MeOH and purged with argon.

6.0 mg (5.7×10³ mmol, 1.2 eq.) of ECI 19 was dissolved in 1 mL phosphate buffer (20 mM, pH 7.4, purged with argon). The EC119 solution was added to the 1-f/MeOH solution while purging with argon. 2 mL DMSO was added to the reaction mixture causing the solution to turn clear. After 5 minutes, LC/MS showed the complete consumption of 1-f. The reaction mixture was diluted with H₂O/DMSO (1:1) to about 9 mL and purified on HPLC with 50 mM NH₄HCO₃ (pH 7.4) and acetonitrile. The fraction containing the desired product was collected, acetonitrile was removed with the rotary evaporator and the aqueous solution was frozen/lyophilized to give 2 mg of the desired product Compound 1 (22%).

LC/MS (ESI-QMS): 966.9 m/z=(M+2H): ¹H NMR (500 MHz, D2O) δ 8.58 (s, 1H), 7.97 (s, 1H), 7.54 (d, J=8.8 Hz, 2H), 7.08-7.10 (m, 2H), 7.03-7.06 (m, 3H), 6.63 (d, J=8.8 Hz, 2H), 5.82 (br, 1H), 5.66 (d, J=11 Hz, 1H), 5.19 (d, J=11 Hz, 1H), 4.57 (t, J=5.8 Hz, 1H), 4.52 (m, 1H), 4.48 (s, 2H), 4.44 (m, 2H), 4.37 (m, 1H), 4.20 (dd, J=8.3, 4.9 Hz, 2H), 4.13 (br, 1H), 3.99 (t, J=6.1 Hz, 1H), 3.82-3.66 (d+br, J=5.9 Hz, 3H), 3.44 (s, 3H), 3.23-3.02 (m, 3H). 3.00 (s, 3H), 2.99-2.93 (m, 4H), 2.80-2.90 (m, 1H), 2.76 (dd, J=13.7, 5.9 Hz, 1H), 2.65-2.62 (m, 1H), 2.58-2.40 (m, 8H), 2.36-2.24 (m, 2H), 2.00-1.90 (m, 3H), 1.90-1.68 (m, 7H), 1.67-1.53 (m, 4H), 1.48-1.32 (m, 4H), 1.32-1.20 (m, 4H), 1.18-1.13 (m, 2H), 1.12-1.02 (m, 1H), 0.98 (d, J=6.8 Hz, 3H), 0.88 (d, J=6.3 Hz, 3H), 0.81 (d, J=6.9 Hz, 3H), 0.74 (t, J=7.3 Hz, 3H), 0.62-0.57 (m, 6H).

### Example 2: Synthesis of Compound 2

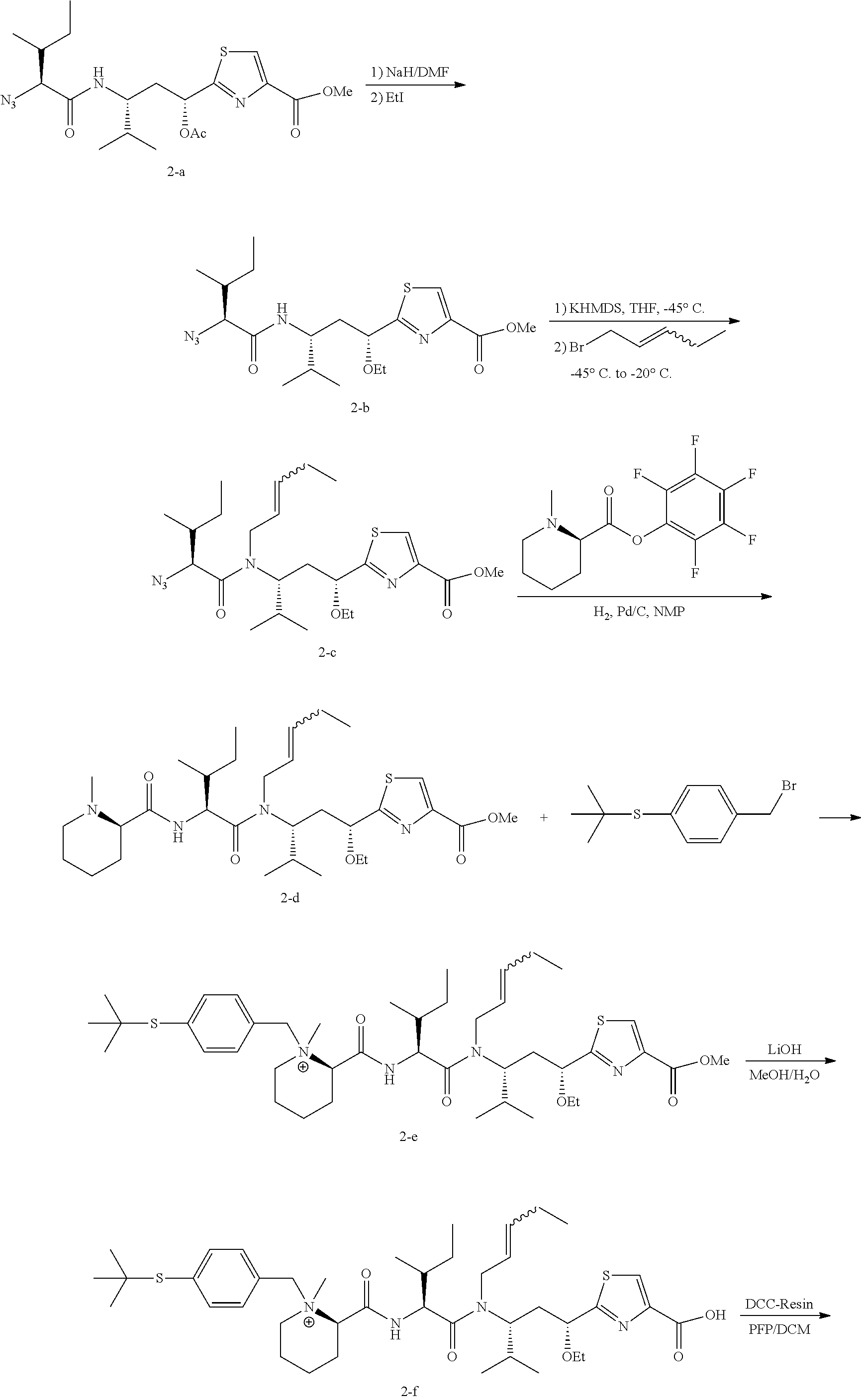

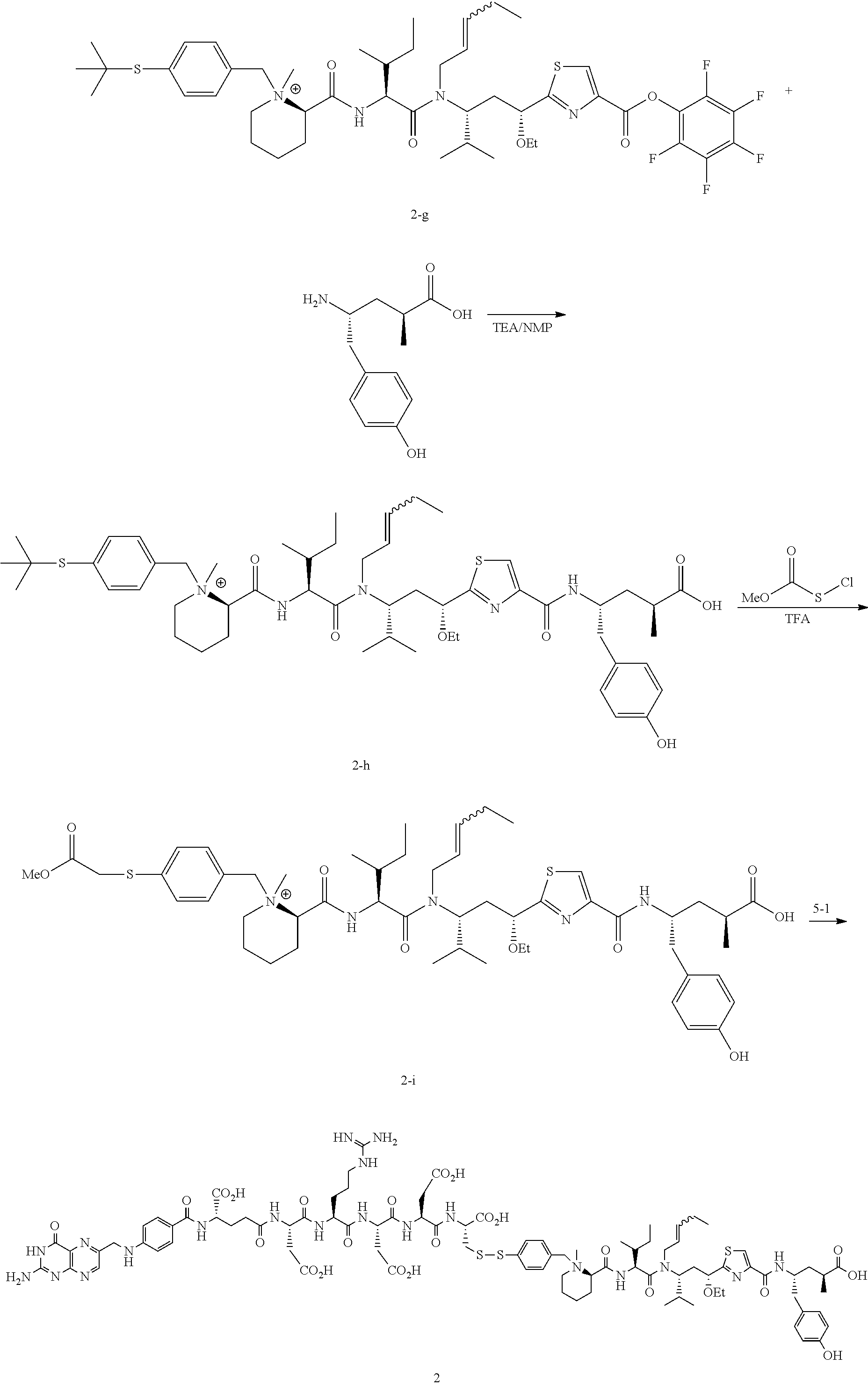

### Step 1: Preparation of 2-b

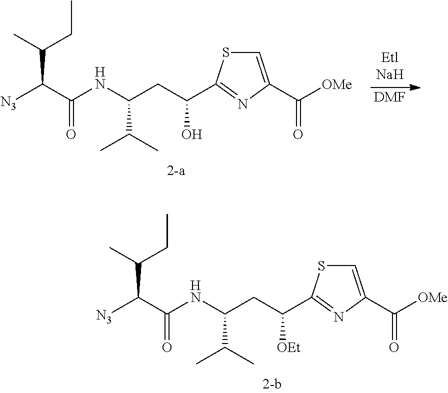

The dipeptide, 2-a (2.22 g, 5.59 mmol, 1 eq.) was dissolved in dimethylformamide (˜10 ml). The solution was cooled to 0° C. (ice-bath) and purged with argon gas. Sodium hydride (227 mg, 5.59 mmol, 60% suspension in oil, 1 eq.) was added in one portion followed by ethyl iodide (491 μL, 6.14 mmol, 1.1 eq.). After 45 minutes, UPLC (10-100% ACN/50 mM pH7 NH₄HCO₃ buffer) showed 86% conversion of the starting material to the desired product with traces of both the hydrolyzed starting material and product also being detected. The reaction mixture was worked up between dichloromethane and brine. The aqueous layer was re-extracted with additional dichloromethane (2×20 mL). The combined organic layers were dried (Na₂SO₄), filtered and the volatiles evaporated in vacuo. The remaining residue was purified by normal phase column chromatography using 0-30-100% EtOAc as the eluent. The product, 2-b was collected as a clear oil (1.7 g, 71%). LC/MS (ESI-QMS): 427.3 m/z=(M+H).

### Step 2: Preparation of 2-c

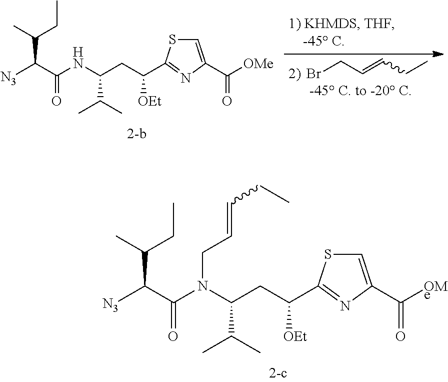

450 mg (1.06 mmol) of 2-b was dissolved in 4 mL anhydrous THF. The solution was cooled to −45° C. KHMDS (0.5 M in toluene, 2.2 mL 1.04 eq.) was added dropwise. The reaction was stirred at −60° C. for 30 minutes. 280 μL of 1-bromo-2-pentene (˜2 eq.) was added to the reaction mixture at −60° C. and the reaction was warmed up over 2 hours to −20° C., and was stored in the freezer (−20° C.) overnight. The reaction was quenched with MeOH, and extracted between EtOAc/H₂O. The organic layers were combined, washed with brine and dried over Na₂SO₄. The salt was filtered off and the solution concentrated leaving behind an oily residue. The residue was purified on a silica column with EtOAc/petroleum ether. The fractions containing the desired product were combined and evaporated to yield 318 mg of compound 2-c (61%).

¹H NMR (500 MHz, CD3OD) for major isomer: δ 8.35 (s, 1H), 5.80-5.63 (m, 2H), 4.60 (br, 1H), 4.02-3.97 (dd, 1H), 3.89 (s, 3H), 3.76 (d, J=9.8 Hz, 1H), 3.55 (t, J=7.0 Hz, 2H), 2.20-2.10 (m, 3H), 2.06-2.00 (m, 2H), 1.80-1.72 (m, 1H), 1.39-1.28 (m, 1H), 1.23 (t, J=7.0 Hz, 3H), 1.01-0.99 (m, 3H), 0.97 (d, J=6.4 Hz, 3H), 0.94 (d, J=6.4 Hz, 3H), 0.89 (d, J=6.3 Hz, 3H).

### Step 3: Preparation of 2-d

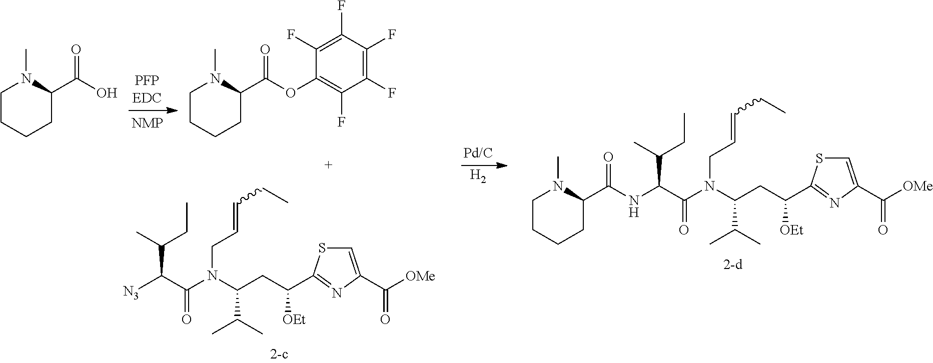

1-N-methyl piperadine 2-carboxylic acid (D-Mep) (232 mg, 1.62 mmol, 4 eq.), pentafluorophenol (298 mg, 1.62 mmol, 4 eq.) and 1-Ethyl-3-(3-dimethylaminopropyl)carbodiimide (310 mg, 1.62 mmol, 4 eq.) were combined in a 50 mL round-bottomed flask under Argon to which 6 mL NMP was added. The reaction mixture was stirred for 4.6 hours after which all solid material had dissolved. 2-d (226 mg, 0.405 mmol, 1 eq.) and Pd/C (43 mg, 10 wt, % loading) were added to the reaction mixture. The reaction vessel was then degassed (under high vacuum) and back-filled with hydrogen gas (balloon) 3 times before stirring the reaction under hydrogen for 3 72 hours. UPLC (10-100% ACN/50 mM pH7 NH₄HCO₃ buffer) showed good conversion to the desired product with traces of the hydrogenated product also being detected. The reaction mixture was quenched with methanol (˜4 mL) and filtered twice through celite. The filter cake was washed with ethyl acetate (4×10 mL) and the filtrate separated between ethyl acetate and a 5% NaHCO₃/10% NaCl (1:1) aqueous solution. The organic extracts were washed with water (2×15 mL) and dried (Na₂SO₄). The solvent was evaporated under reduced pressure and the remaining crude material (440 mg) purified on a 12 g silica column using 0-30-100% EtOAc/Petroleium ether as the eluent. The product, compound 2-d, was collected as a clear oil (181 mg, 67%). LC/MS (ESI-QMS): 593.0 m/z=(M+H).

### Step 4: Preparation of 2-e

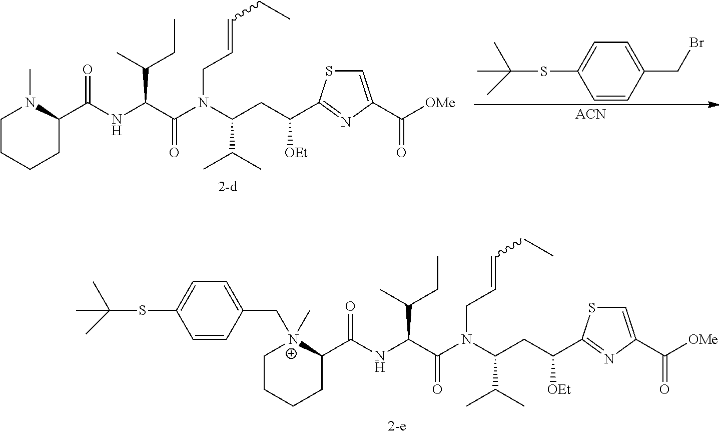

Compound 2-d (50 mg, 0.17 mmol, 1 eq.) was dissolved in acetonitrile (˜3 ml) to which 2-[(4-(bromomethyl)phenyl)sulfanyl]-2-methylpropane (131 mg, 0.506 mmol, 3 eq.) was added. The reaction was stirred under an argon atmosphere overnight. TLC (10% MeOH/DCM) showed the reaction had gone to completion. The solvent was removed under reduced pressure and the remaining residue purified on a 12 g silica column using 0-10% MeOH/DCM as the eluent. The desired product, compound 2-e, was collected as a mixture (52 mg) which contained 5% of the starting material, 2-d. LC/MS (ESI-QMS): 771.6 m/z=(M+H).

### Step 5: Preparation of 2-f

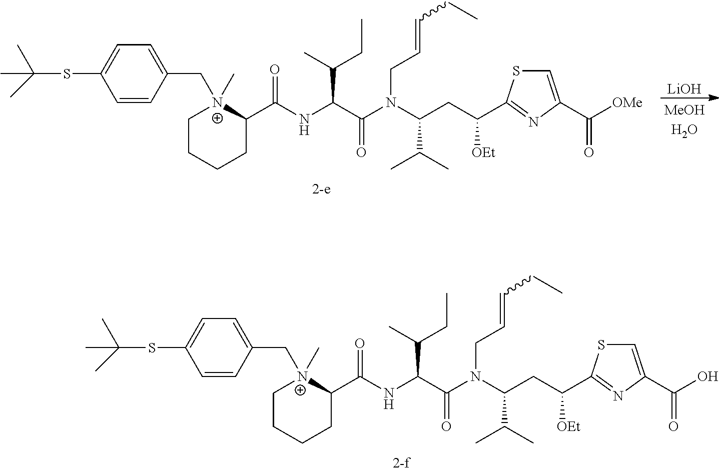

18 mg of 2-e (0.023 mmol) was dissolved in 1 mL MeOH, and LiOH H₂O (42 μL of a 137 mg of LiOH.H₂O in 3 mL H₂O solution, 2 eq.) was added to the solution. The reaction was monitored by LC/MS. Additional LiOH H₂O solution was added to force the reaction to go to completion. In total, 126 μL of the LiOH.H₂O solution was used (6 eq.). The solvents were removed under reduced pressure and the reaction mixture was pumped on high vacuum to dryness. DCM was used to dissolve the organic compound and the insoluble materials were removed by centrifuge. 10 mg of crude material was recovered and used without further purification. The identity of compound 2-f was confirmed by LC/MS (ESI) [M]⁺757.35.

### Step 6 and Step 7: Preparation of 2-g and 2-h

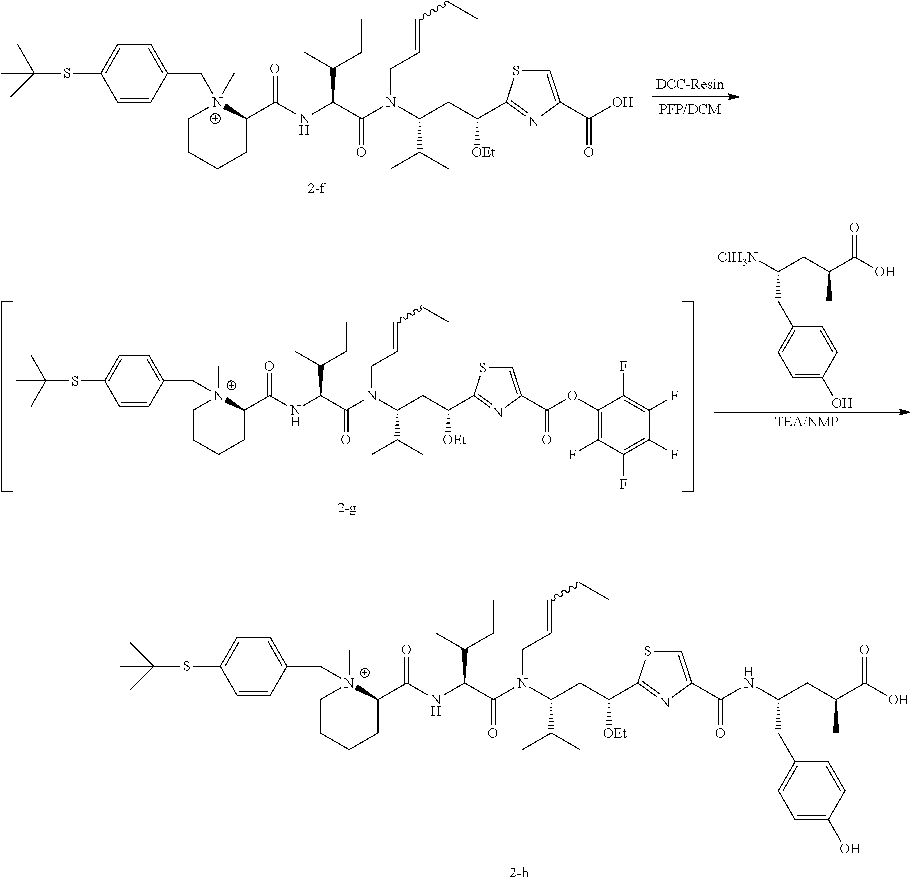

10 mg of the compound 2-f (0.013 mmol) was mixed with 5 mg of PFP (0.027 mmol, 2 eq.) and 60 mg of DCC-resin (0.138 mmol, 10 eq.) in 1 mL DCM. The reaction mixture was stirred at room temperature. LC/MS showed there was some conversion overnight. Another 60 mg of DCC-resin was added to the reaction mixture and the reaction mixture was stirred over 2 days. LC/MS showed the presence of activated acid. The resin was filtered off using a syringe filter and washed with DCM. The combined solution was concentrated under reduced pressure. 3.5 mg of Tut-HCl with 37 μL of TEA (20 eq.) were dissolved in 0.3 mL DMSO and added to the activated acid residue. The reaction was monitored by LC/MS. After the reaction stopped progressing, the reaction mixture was diluted with EtOAc, and extracted with H₂O. The organic layer was separated and evaporated to dryness to afford 15 mg of the crude product, containing compound 2-h, compound 2-f, and PFP. The identity of compound 2-h was confirmed by LC/MS (ESI) [M]⁺962.54. Crude compound 2-h from Step 7 was used without further purification. The amounts of reagents required for the next step were calculated based on 10 mg of 2-f (0.013 mmol).

### Step 8 and Step 9: Preparation of Compounds 2-i and 2

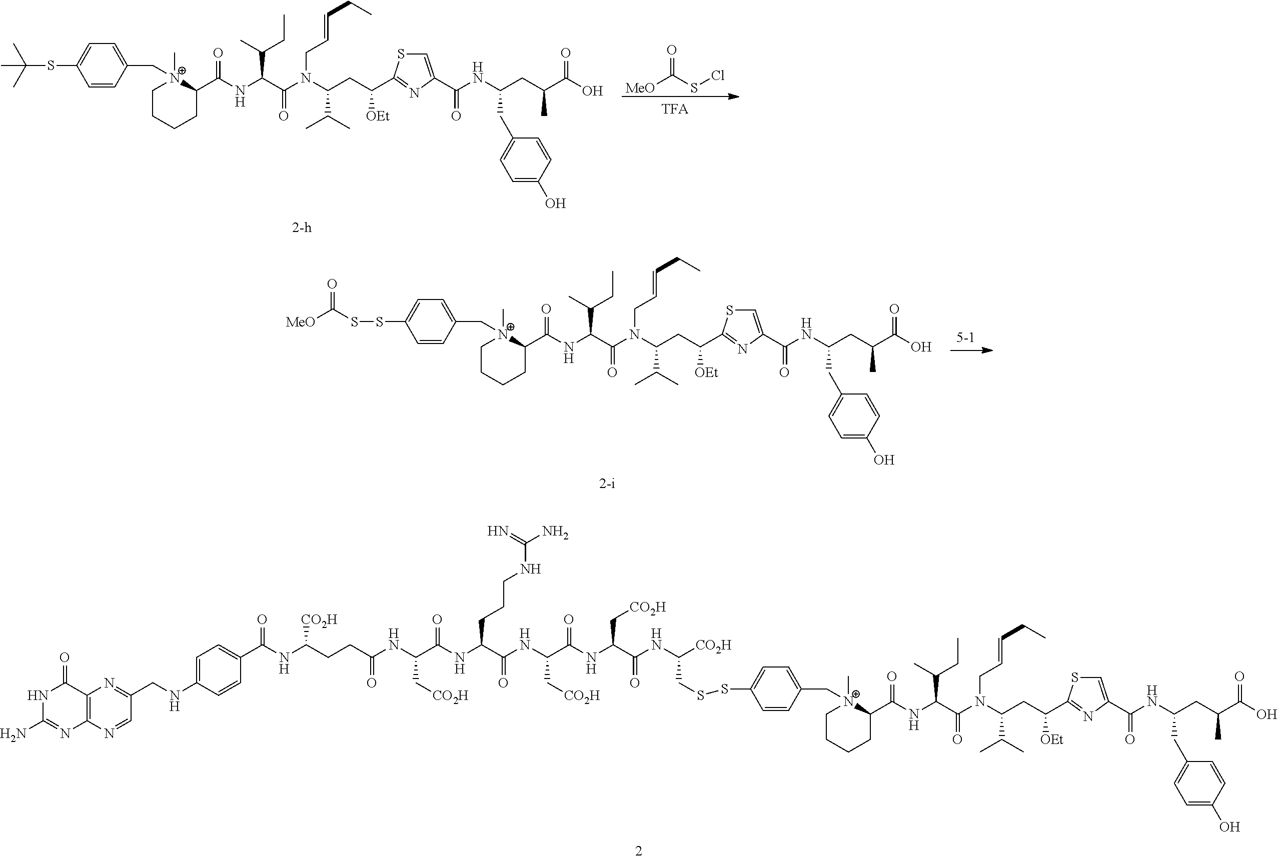

Crude compound 2-h was mixed with 24 μL of methoxycarbonylsulfenyl chloride (0.265 mmol, 20 eq.), and 0.3 mL of TFA was added to the mixture. The reaction mixture was stirred at room temperature and monitored by LC/MS. After 10 minutes, the starting material was consumed, and the formation of the desired product, along with a range of decomposition side-products was observed. TFA and methoxycarbonylsulfenyl chloride were removed under high vacuum, and the resulting residue was used without further purification.

14 mg of 5-1 (0.013 mmol) was dissolved in 2 mL of 20 mM phosphate buffer (purged with argon). The solution of 5-1 was added to the above tubulysin-disulfide residue in 0.5 mL DMSO. The reaction mixture was stirred at room temperature and purged with argon. 2 mL of MeOH was added to the reaction mixture, forming a homogeneous solution. The reaction resulted in the UV detection of 4 major peaks. The reaction mixture was diluted with 20 mM phosphate buffer to about 9 mL and was purified on HPLC with 50 mM NH₄HCO₃ buffer (pH 7.4) and acetonitrile as eluents. The fraction containing compound 2 was collected, and acetonitrile removed under reduced pressure. The aqueous solution was frozen and lyophilized to afford 0.7 mg (3% yield over 4 steps) of EC2971. This was confirmed by LC/MS (ESI) [M+2H]²+977.05. Selected signals for NMR: ¹H NMR (500 MHz, D₂O): δ 8.63 (s, 1H), 7.22 (d, J=7.5 Hz, 2H), 6.63 (d, J=7.5 Hz, 2H), 5.69 (br, 1H), 5.30 (br, 1H).

### Chemical Release Examples

### Chemical Release Example 1: Release Study of Compound 1

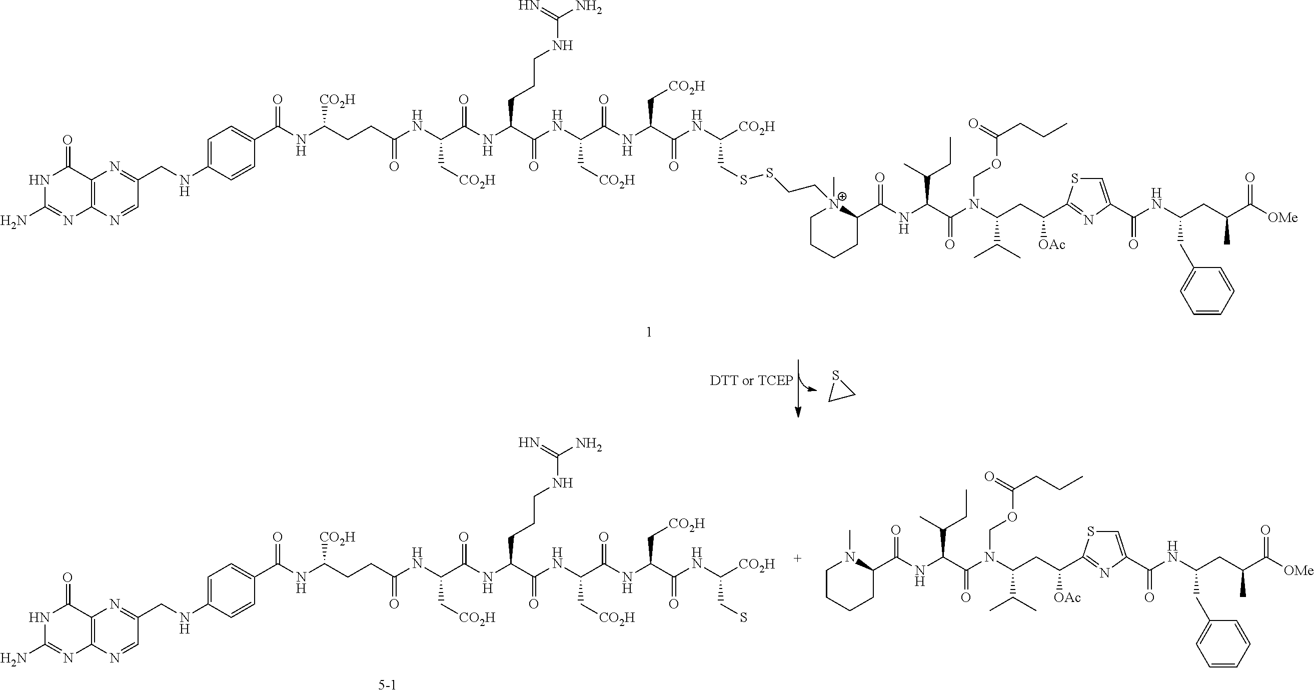

After the HPLC purification, a small portion of the fraction containing compound 1 was subjected to an excess of DTI or TCEP solution (0.5 M, neutral). The fraction contained 50 mM NH₄HCO₃ (pH 7.4) and acetonitrile (ratio ˜1:1). The release profiles were recorded on UPLC/MS. The identity of the freed tubulysin was confirmed by comparison against the retention time and MS of an authentic sample.

### Biological Examples

### Biological Example 1: In Vitro FR Specific Activity of Folate Conjugates

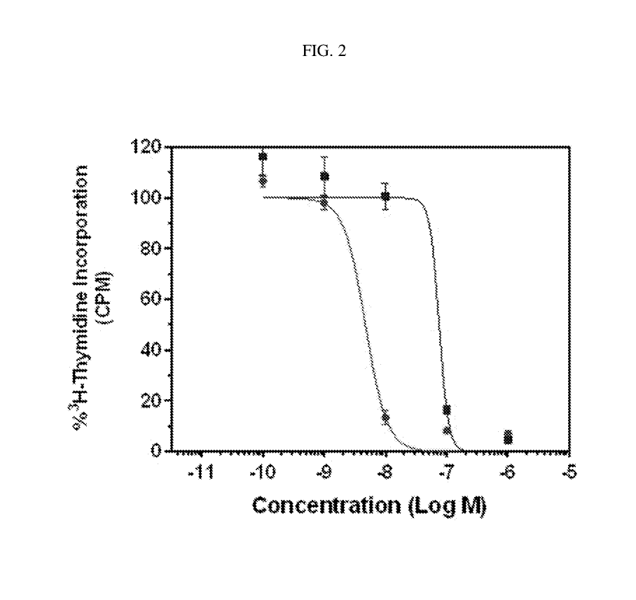

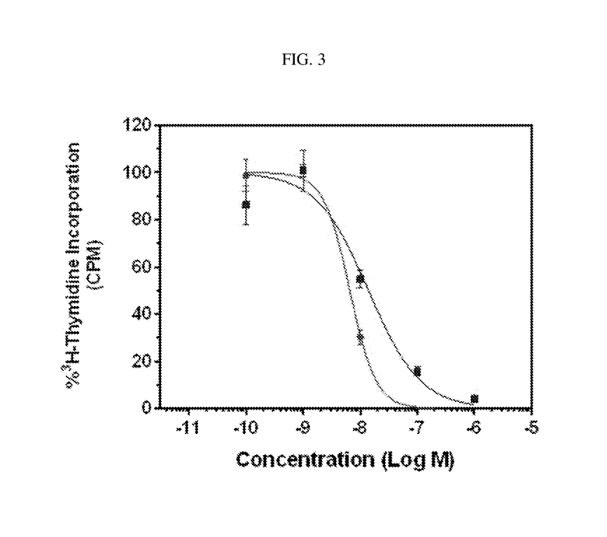

KB cells were seeded in individual 24-well Falcon plates and allowed to form nearly confluent monolayers overnight in FFRPMI/HIFCS. Thirty minutes prior to the addition of folate-conjugate, spent medium was aspirated from all wells and replaced with either fresh FFRPMI or FFRPMI supplemented with 100 μM FA. Each well then received 1 mL of medium containing increasing concentrations of folate-conjugate (3 wells per sample). Cells were pulsed for 2 h at 37° C., rinsed 4 times with 0.5 mL of medium and then chased in 1 mL of fresh medium up to 72 h. Spent medium was aspirated from all wells and replaced with fresh medium containing 5 μCi/mL of ³H-thymidine. Following a 2 h incubation at 37° C., cells were washed 3 times with 0.5 mL of PBS and then treated with 0.5 mL of ice-cold 5% trichloroacetic acid per well. After 15 min, the trichloroacetic acid was aspirated and the cells solubilized by the addition of 0.5 mL of 0.25 N sodium hydroxide for 15 min at room temperature. Four hundred and fifty μL of each solubilized sample were transferred to scintillation vials containing 3 mL of Ecolume scintillation cocktail and counted in a liquid scintillation counter. Final results were expressed as the percentage of ³H-thymidine incorporation relative to untreated controls. FIG. 2 shows results using Compound 1 and Compound 1+excess folate. FIG. 3 shows results using Compound 2 and Compound 2+excess folate.

We claim:

1. A drug conjugate comprising a binding ligand, a linker, and a tertiary amine containing drug, wherein the linker comprises a disulfide bond, the binding ligand is covalently attached to the linker, and the tertiary amine containing drug is covalently attached to the linker through a nitrogen atom of a tertiary amine group on the tertiary amine containing drug, such that the drug conjugate contains a quaternary amine.

2. The drug conjugate of claim 1, or a pharmaceutically acceptable salt thereof, wherein linker comprises a moiety L¹ of the formula selected from the group consisting of 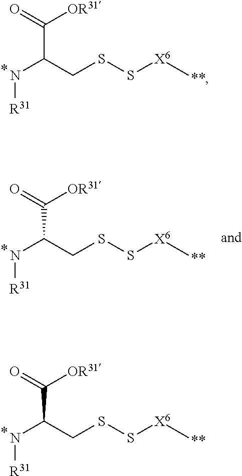
wherein
each of R³¹ and R^{31′} is independently selected from the group consisting of H, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R³², —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{30′}, —C(O)R³², —C(O)OR³² or —C(O)NR³²R^{32′};
X⁶ is independently a C₁-C₆ alkyl, C₂-C₆ heteroalkyl or C₆-C₁₀ aryl, wherein each hydrogen atom in C₁-C₆ alkyl, C₁-C₆ heteroalkyl and C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{30′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁰R^{30′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R³², R^{32′}, R³³, R^{33′}, R³⁴, R^{34′}, R³⁵ and R^{35′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
wherein ** is a covalent bond to a nitrogen atom of a tertiary amine on the tertiary amine containing drug; and * is a covalent bond to the rest of the drug conjugate.

3. The drug conjugate of claim 2, or a pharmaceutically acceptable salt thereof, wherein X⁶ is C₁-C₆ alkyl; wherein each hydrogen atom in C₁-C₆ alkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}.

4. The drug conjugate of claim 3, or a pharmaceutically acceptable salt thereof, wherein X⁶ is methyl, ethyl, n-propyl, iso-propyl, n-butyl, sec-butyl or n-pentyl.

5. The drug conjugate of claim 2, or a pharmaceutically acceptable salt thereof, wherein X⁶ is C₁-C₆ heteroalkyl; wherein each hydrogen atom in C₁-C₆ heteroalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³, —OC(O)R³⁴, —OC(O)NR³⁴R^{34′}, —OS(O)R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —NR³⁴R^{34′}, —NR³⁴C(O)R³⁵, —NR³⁴C(O)OR³⁵, —NR³⁴C(O)NR³⁵R^{35′}—NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R³⁴.

6. The drug conjugate of claim 5, or a pharmaceutically acceptable salt thereof, wherein C₁-C₆ heteroalkyl comprises one heteroatom selected from the group consisting of N, O and S.

7. The drug conjugate of claim 2, or a pharmaceutically acceptable salt thereof, wherein X⁶ is C₆-C₁₀ aryl, wherein each hydrogen atom in C₆-C₁₀ aryl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}.

8. The drug conjugate of claim 7, or a pharmaceutically acceptable salt thereof, wherein C₆-C₁₀ aryl is phenyl.

9. The drug conjugate of claim 1, or a pharmaceutically acceptable salt thereof, wherein the tertiary amine containing drug is selected from the group consisting of an opioid, an antibiotic, an antidepressant and a cancer therapeutic.

10. The drug conjugate of claim 1, or a pharmaceutically acceptable salt thereof, wherein the tertiary amine containing drug is selected from the group consisting of morphine, hydrocodone, oxycodone, codeine, mitragynol, vinblastine, vincristine, vindesine, vinorelbine, clindamycin, novobiocin, retapamulin, dimethylpipBOR, N,N-dimethylsitafloxacin, rifampin, azithromycin, venlafaxine, mirtazapine, escitalopram, porfiromycin, pamamycin 601, macromerine, tatreponerine 8, imatinib, aripiprazole, buprenorphine, sildenafil, quetiapine, methylphenidate, doxycycline, solifenacin, lidocaine, eszopiclone, and tubulysin.

11. The drug conjugate of claim 1, or a pharmaceutically acceptable salt thereof, wherein the linker comprises at least one AA selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

12. The drug conjugate of claim 11, or a pharmaceutically acceptable salt thereof, wherein the linkers comprises at least one AA selected from the group consisting of L-arginine, L-aspartic acid, L-cysteine, D-arginine, D-aspartic acid, and D-cysteine.

13. The drug conjugate of claim 1, or a pharmaceutically acceptable salt thereof, wherein the linker further comprises at least one spacer linker (L²) of the formula 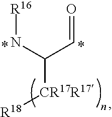
wherein
R¹⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R², —OC(O)NR²⁰R²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R²⁵, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, (CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R³⁰, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5; and
q is 1, 2, 3, 4 or 5;
wherein each * is a covalent bond to the rest of the drug conjugate.

14. The drug conjugate of claim 13, or a pharmaceutically acceptable salt thereof, wherein R¹⁶ is H.

15. The drug conjugate of claim 13, or a pharmaceutically acceptable salt thereof, wherein R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5; and
q is 1, 2, 3, 4 or 5;
wherein each * is a covalent bond to the rest of the drug conjugate.

16. The drug conjugate of claim 13, or a pharmaceutically acceptable salt thereof, wherein R¹⁸ is selected from the group consisting of H, 5- to 7-membered heteroaryl, —OR²⁶, NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹,
each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;
R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);
R²⁸ is H or sugar;
n is 1, 2, 3, 4 or 5;
p is 1, 2, 3, 4 or 5; and
q is 1, 2, 3, 4 or 5;
wherein * is a covalent bond to the rest of the drug conjugate.

17. The drug conjugate of claim 13, or a pharmaceutically acceptable salt thereof, wherein each L² is independently selected from the group consisting of 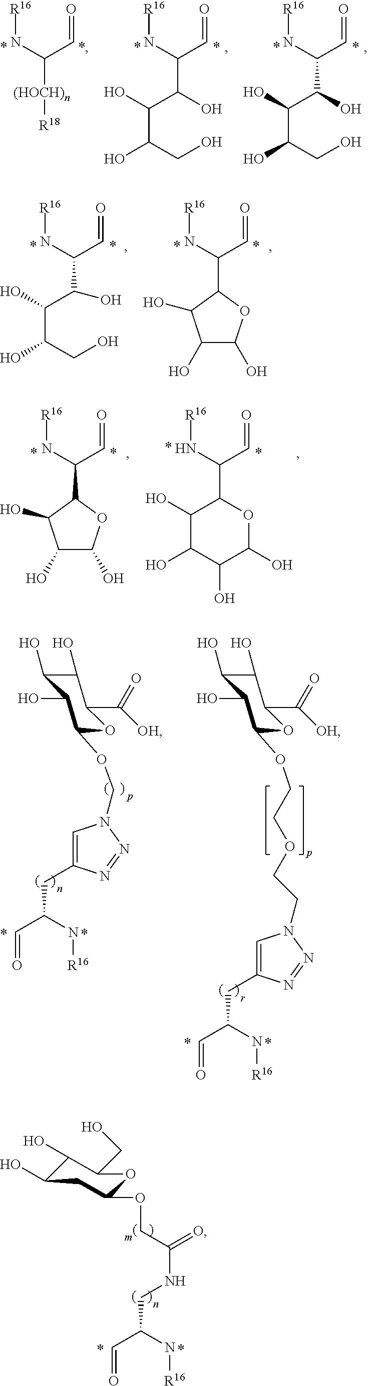 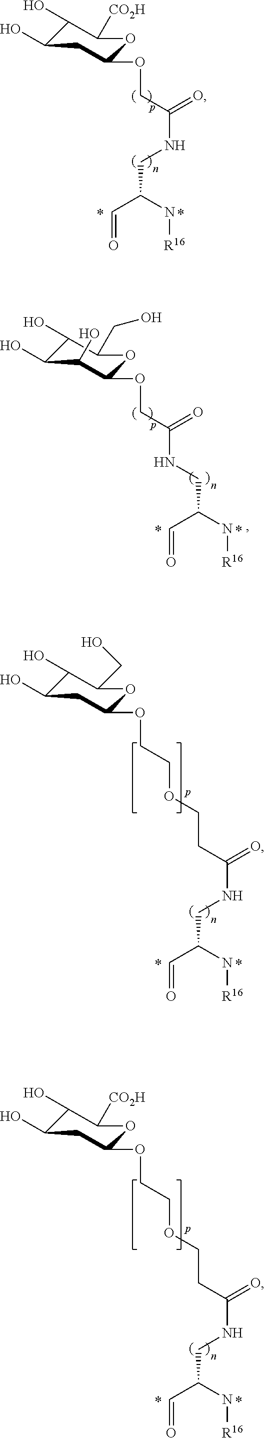 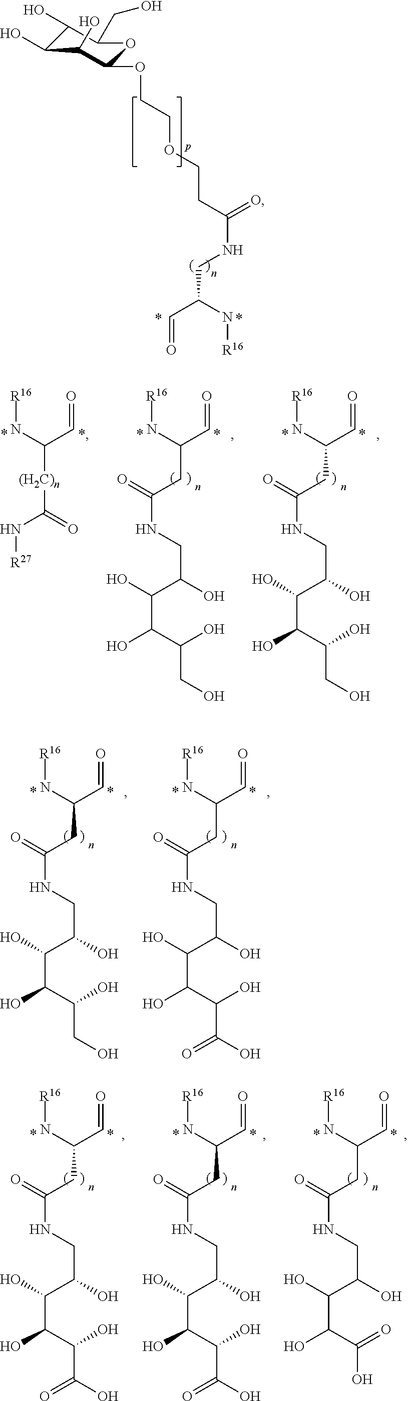 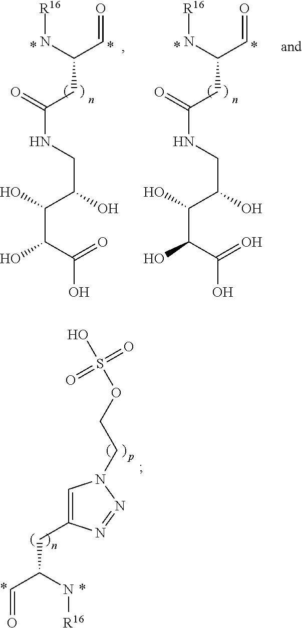
and combinations thereof,
wherein
R⁶ is selected from the group consisting of H, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁶, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each each R¹⁹, R^{19′}, R²⁰, R^{20′}, R²¹, R^{21′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C1-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5; and
q is 1, 2, 3, 4 or 5;
wherein each * is a covalent bond to the rest of the drug conjugate.

18. The drug conjugate of claim 13, or a pharmaceutically acceptable salt thereof, wherein each L² is selected from the group consisting of 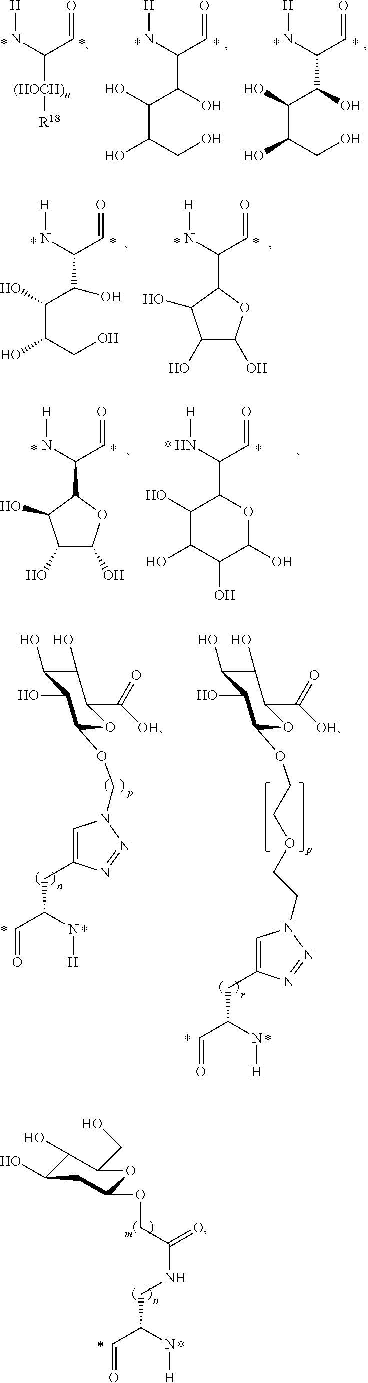 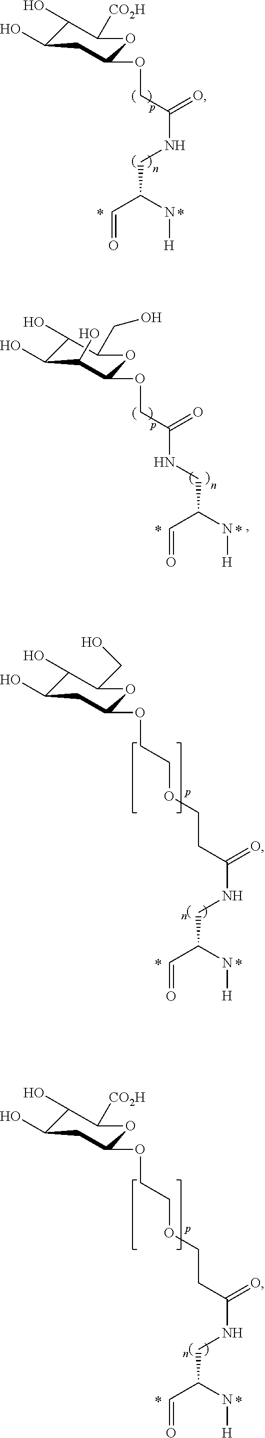 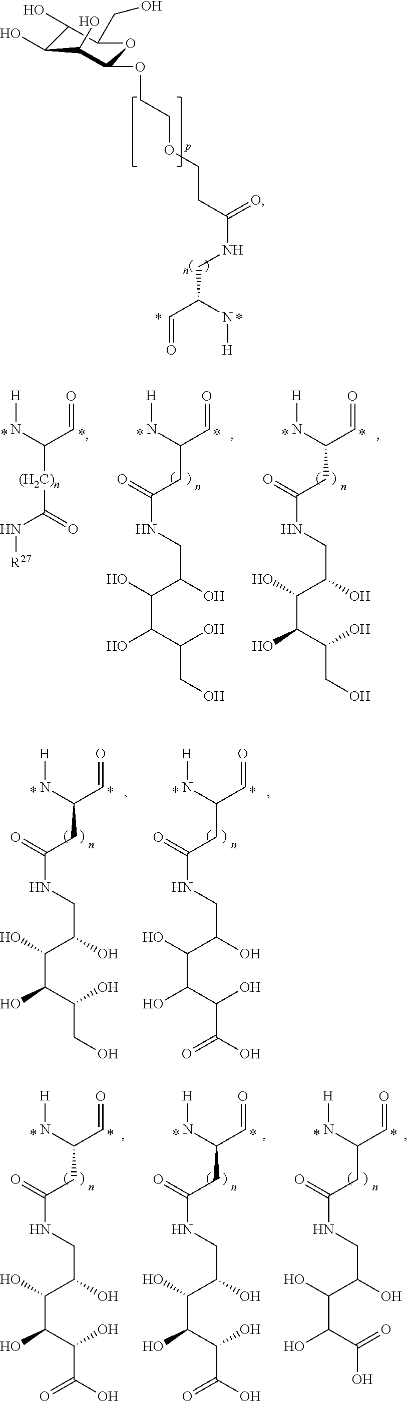 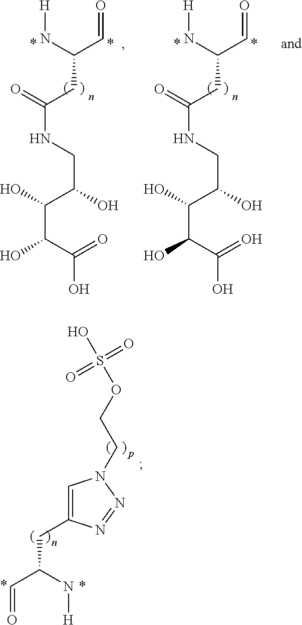
and combinations thereof,
wherein
R¹⁸ is selected from the group consisting of H, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C1-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5; and
q is 1, 2, 3, 4 or 5;
wherein each * is a covalent bond to the rest of the drug conjugate.

19. The drug conjugate of claim 1, or a pharmaceutically acceptable salt thereof, wherein the binding ligand is of the formula 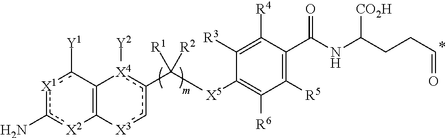
wherein
R¹ and R² in each instance are independently selected from the group consisting of H, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, C₁-C₆ alkyl, C₂-C₆ alkenyl or C₂-C₆ alkynyl;
X¹ is —NR¹¹—, ═N—, —N═, —C(R¹¹)═ or —C(R¹¹)—;
X² is —NR¹¹— or ═N—;
X³ is —NR^{11″}—, —N═ or —C(R^{11′})═;
X⁴ is —N═ or —C≡;
X⁵ is NR¹² or CR¹²R^{12′};
Y¹ is H, —OR¹³, —SR¹³ or —NR¹³R^{13′} when X¹ is —N═ or —C(R¹¹)═, or Y¹ is ═O when X¹ is —NR¹¹—, ═N— or —C(R¹¹)—;
Y² is H, C₁-C₆ alkyl, C₂-C₆ alkenyl, —C(O)R¹⁴, —C(O)OR¹⁴, —C(O)NR¹⁴R^{14′} when X⁴ is —C═, or Y² is absent when X⁴ is —N═;
R¹¹, R^{11′}, R^{11″}, R¹², R^{12′}, R¹³, R^{13′}, R¹⁴ and R^{14′} are each independently selected from the group consisting of H, C₁-C₆ alkyl, —C(O)R¹⁵, —C(O)OR¹⁵ and —C(O)NR¹⁵R^{15′};
R¹⁵ and R^{15′} are each independently H or C₁-C₆ alkyl; and
m is 1, 2, 3 or 4;
wherein * is a covalent bond to the rest of the drug conjugate.

20. The drug conjugate of claim 19, wherein B is of the formula 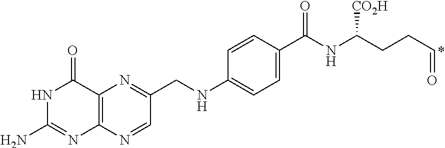 [SMILES: Nc1nc2ncc(CNc3ccc(C(=O)N[C@@H](CCC=O)C(=O)O)cc3)nc2c(=O)[nH]1]
wherein * is a covalent bond to the rest of the drug conjugate.
